(19) United States
(12) Patent Application Publication
(10) Pub. No.: US 2022/0162200 A1
(43) Pub. Date: May 26, 2022

(54) PKM2 MODULATORS AND METHODS FOR THEIR USE

(71) Applicant: Sumitomo Dainippon Pharma Oncology, Inc., Cambridge, MA (US)

(72) Inventor: Adam Siddiqui-Jain, South Jordan, UT (US)

(21) Appl. No.: 17/439,611

(22) PCT Filed: Mar. 20, 2020
(86) PCT No.: PCT/US2020/024035

§ 371 Date: Sep. 15, 2021

Related U.S. Application Data
(60) Provisional application No. 62/822,700, filed on Mar. 22, 2019.

(51) Int. Cl. C07D 409/12; C07D 409/14

(52) U.S. Cl. CPC C07D 409/12; C07D 409/14

(57) ABSTRACT

Compounds having activity as PKM2 activators are disclosed. The compounds have the following structure (I), including pharmaceutically acceptable salts, isotopic forms, tautomers and prodrugs thereof, wherein R1, R2, and R3 are as defined herein. Methods associated with preparation and use of such compounds, as well as pharmaceutical compositions comprising such compounds, are also disclosed.

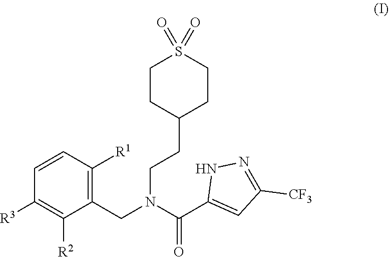

### FIELD

The present disclosure relates to compounds, compositions comprising such compounds, and their use for the treatment of PKM2 mediated diseases or disorders.

### BACKGROUND

PKM2 is upregulated in cancer cells (Altenberg B., Greulich K. O., Genomics 84(6):1014-20 (2004)) and has been shown to increase tumorigenicity compared to the alternatively spliced and constitutively active PKM1 isoform (Christofk H. R., Vander Heiden M. G., Harris M. H., et al., Nature 452(7184):230-33 (2008); Goldberg M. S., Sharp P. A., J. Exp. Med. 209(2):217-24 (2012)). The shift from PKM1 to PKM2 metabolically reprograms cells to create an environment where the tumorigenic cells are able to balance their energetic needs with their requirements for biomolecular building blocks to support cell growth. Cancer cells shift their dependence from PKM1 to PKM2 through multiple mechanisms, including oncoprotein binding (Kosugi M., Ahmad R., Alam M., Uchida Y., Kufe D., PLoS One 6(11):e28234 (2011); Zwerschke W., Mazurek S., Massimi P., Banks L., Eigenbrodt E., Jansen-Durr P., Proc. Nat'l Acad. Sci. U.S.A. 96(4):1291-96 (1999)), tyrosine phosphorylation (Hitosugi T., Kang S., Vander Heiden M. G., et al., Sci. Signal 2(97):ra73 (2009); Presek P., Glossmann H., Eigenbrodt E., et al., Cancer Res. 40(5):1733-41 (1980); Presek P., Reinacher M., Eigenbrodt E., FEBS Lett. 242(1):194-98 (1988)), lysine acetylation (Lv L., Li D., Zhao D., et al., Mol. Cell 42(6):719-30 (2011)), cysteine oxidation (Anastasiou D., Poulogiannis G., Asara J. M., et al., Science 334(6060):1278-83 (2011)), and prolyl hydroxylation (Chen N., Rinner O., Czernik D., et al., Cell Res. 21(6):983-86 (2011)). In each case, PKM2 activity correlates with increased tumorigenicity. As a partially active enzyme, PKM2 creates an opportunity where both small molecule PKM2 activators (Boxer M. B., Jiang J. K., Vander Heiden M. G., et al., J Med. Chem. 53(3):1048-55 (2010); Jiang J. K., Boxer M. B., Vander Heiden M. G., et al., Bioorg. Med. Chem. Lett. 20(11):3387-93 (2010); Walsh M. J., Brimacombe K. R., Veith H., et al., Bioorg. Med. Chem. Lett. 21(21):6322-27 (2011)) or inhibitors could potentially disrupt the metabolic balance that cancer cells require. Therefore, both PKM2 activators and inhibitors have been proposed to be useful anti-cancer therapies. The present disclosure focuses on activators of PKM2.

### SUMMARY

There remains a need for new treatments and therapies for PKM2 mediated disorders or diseases. The present disclosure provides compounds and pharmaceutical compositions thereof, which compounds are PKM2 modulators. The present disclosure further provides methods of treating PKM2 mediated disorders or diseases, comprising administering to a subject in need thereof a therapeutically effective amount of a PKM2 modulator, for example a PKM2 activator. In one embodiment, compounds having the following structure (I) are provided:

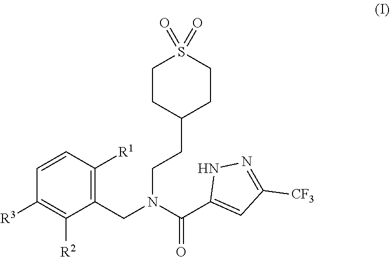

including pharmaceutically acceptable salts, isotopic forms, stereoisomers, tautomers, and prodrugs thereof, wherein R¹, R², and R³ are as defined herein.

In another embodiment, a pharmaceutical composition is provided comprising a compound having structure (I), or a pharmaceutically acceptable salt, isotopic form, stereoisomer, tautomer, or prodrug thereof, and a pharmaceutically acceptable carrier, diluent or excipient.

In another embodiment, a method for modulating PKM2 in a subject in need thereof is provided, the method comprising administering to the subject a therapeutically effective amount of a compound having structure (I), or a pharmaceutically acceptable salt, isotopic form, stereoisomer, tautomer, or prodrug thereof. In some embodiments, modulation of PKM2 comprises activating PKM2. In some embodiments the method is for treatment of cancer.

### DETAILED DESCRIPTION

Various (enumerated) embodiments of the disclosure are described herein. It will be recognized that features specified in each embodiment may be combined with other specified features to provide further embodiments of the present disclosure.

### Embodiment 1

A compound having the following structure (I):

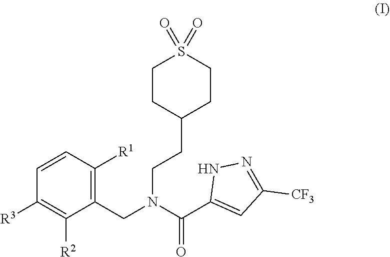

or a pharmaceutically acceptable salt, isotopic form, tautomer or prodrug thereof, wherein:

R¹ is F or NH₂,

R² is H, Cl or Br; or R² joins with R³ to form an optionally substituted heteroaryl; and

R³ is H or optionally substituted C₁-C₆ alkyl; or R³ joins with R² to form an optionally substituted heteroaryl.

### Embodiment 2

The compound of Embodiment 1, wherein:

R¹ is F or NH₂,

R² is H, Cl or Br; and

R³ is H or optionally substituted C₁-C₆ alkyl.

### Embodiment 3

The compound of Embodiment 1 or 2, wherein R³ is H.

### Embodiment 4

The compound of Embodiment 1 or 2, wherein R³ is optionally substituted C₁-C₆ alkyl.

### Embodiment 5

The compound of Embodiment 4, wherein the optionally substituted C₁-C₆ alkyl is methyl or aminomethyl.

### Embodiment 6

The compound of any one of Embodiments 1-5, wherein R² is H.

### Embodiment 7

The compound of Embodiment 1, wherein:

R¹ is F or NH₂,

R² is Cl or Br; and

R³ is H.

### Embodiment 8

The compound of Embodiment 7, wherein R¹ is F.

### Embodiment 9

The compound of Embodiment 7, wherein R¹ is NH₂.

### Embodiment 10

The compound of Embodiment 7, wherein R² is Cl.

### Embodiment 11

The compound of Embodiment 7, wherein R² is Br.

### Embodiment 12

The compound of Embodiment 1, wherein R² and R³ join to form an optionally substituted heteroaryl.

### Embodiment 13

The compound of Embodiment 12, wherein the optionally substituted heteroaryl is a 5-membered heteroaryl.

### Embodiment 14

The compound of Embodiment 12 or 13, wherein R¹ is F.

### Embodiment 15

The compound of Embodiment 12 or 13, wherein R¹ is NH₂.

### Embodiment 16

A compound having the following structure (Ia):

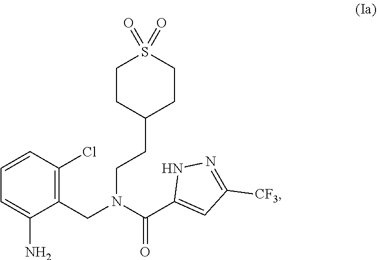 [SMILES: Nc1cccc(Cl)c1CN(CCC1CCS(=O)(=O)CC1)C(=O)c1cc(C(F)(F)F)n[nH]1]

or a pharmaceutically acceptable salt, isotopic form, tautomer or prodrug thereof.

### Embodiment 17

A compound having the following structure (Ib):

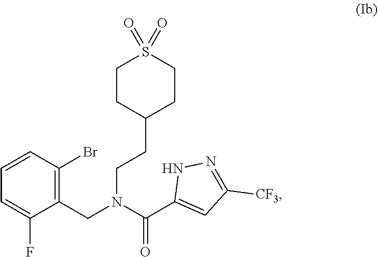 [SMILES: O=C(c1cc(C(F)(F)F)n[nH]1)N(CCC1CCS(=O)(=O)CC1)Cc1c(F)cccc1Br]

or a pharmaceutically acceptable salt, isotopic form, tautomer or prodrug thereof.

### Embodiment 18

A compound having the following structure (Ic):

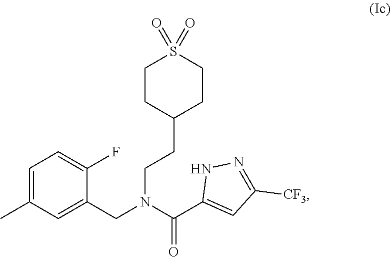 [SMILES: Cc1ccc(F)c(CN(CCC2CCS(=O)(=O)CC2)C(=O)c2cc(C(F)(F)F)n[nH]2)c1]

or a pharmaceutically acceptable salt, isotopic form, tautomer or prodrug thereof.

### Embodiment 19

A compound having the following structure (Id):

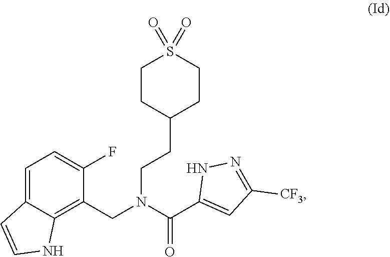 [SMILES: O=C(c1cc(C(F)(F)F)n[nH]1)N(CCC1CCS(=O)(=O)CC1)Cc1c(F)ccc2cc[nH]c12]

or a pharmaceutically acceptable salt, isotopic form, tautomer or prodrug thereof.

### Embodiment 20

A compound having the following structure (Ie):

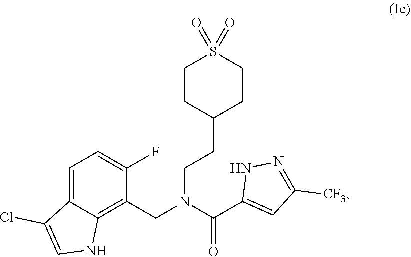 [SMILES: O=C(c1cc(C(F)(F)F)n[nH]1)N(CCC1CCS(=O)(=O)CC1)Cc1c(F)ccc2c(Cl)c[nH]c12]

or a pharmaceutically acceptable salt, isotopic form, tautomer or prodrug thereof.

### Embodiment 21

A compound having the following structure (If):

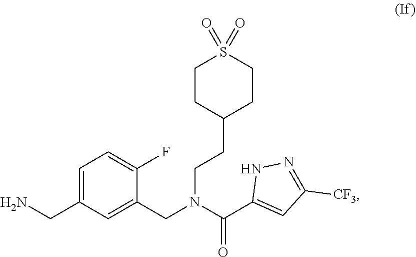 [SMILES: NCc1ccc(F)c(CN(CCC2CCS(=O)(=O)CC2)C(=O)c2cc(C(F)(F)F)n[nH]2)c1]

or a pharmaceutically acceptable salt, isotopic form, tautomer or prodrug thereof.

### Embodiment 22

A compound having the following structure (Ig):

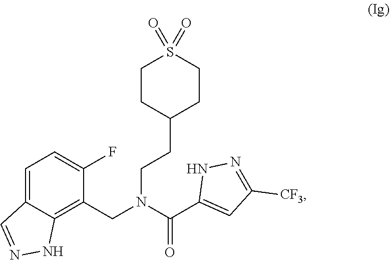 [SMILES: O=C(c1cc(C(F)(F)F)n[nH]1)N(CCC1CCS(=O)(=O)CC1)Cc1c(F)ccc2cn[nH]c12]

or a pharmaceutically acceptable salt, isotopic form, tautomer or prodrug thereof.

### Embodiment 23

A compound having the following structure (Ih):

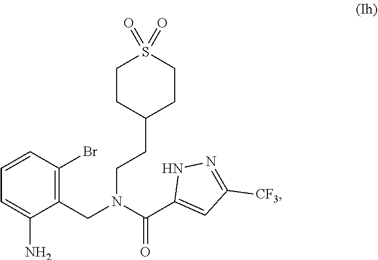 [SMILES: Nc1cccc(Br)c1CN(CCC1CCS(=O)(=O)CC1)C(=O)c1cc(C(F)(F)F)n[nH]1]

or a pharmaceutically acceptable salt, isotopic form, tautomer or prodrug thereof.

### Embodiment 24

A compound having the following structure (Ii):

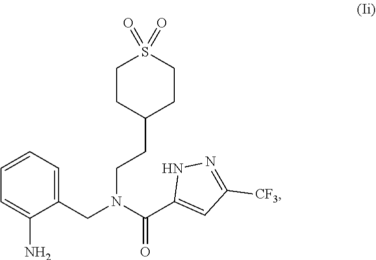 [SMILES: Nc1ccccc1CN(CCC1CCS(=O)(=O)CC1)C(=O)c1cc(C(F)(F)F)n[nH]1]

or a pharmaceutically acceptable salt, isotopic form, tautomer or prodrug thereof.

### Embodiment 25

A compound having the following structure (Ij):

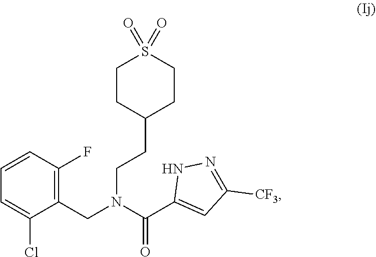 [SMILES: O=C(c1cc(C(F)(F)F)n[nH]1)N(CCC1CCS(=O)(=O)CC1)Cc1c(F)cccc1Cl]

or a pharmaceutically acceptable salt, isotopic form, tautomer or prodrug thereof.

### Embodiment 26

A pharmaceutical composition comprising the compound of any one of Embodiments 1-25, or a pharmaceutically acceptable salt, isotopic form, tautomer or prodrug thereof, and a pharmaceutically acceptable carrier or excipient.

### Embodiment 27

The pharmaceutical composition of Embodiment 26, wherein the pharmaceutical composition is formulated for oral administration.

### Embodiment 28

The pharmaceutical composition of Embodiment 26, wherein the pharmaceutical composition is formulated for injection.

### Embodiment 29

A method for modulating pyruvate kinase muscle isozyme M2 (PKM2) activity in a subject in need thereof, the method comprising administering a therapeutically effective amount of the compound of any one of Embodiments 1-25, or a tautomer, pharmaceutically acceptable salt or prodrug thereof, or the composition of any one of Embodiments 26-28 to the subject.

### Embodiment 30

The method of Embodiment 29, wherein the modulating comprises activating PKM2.

### Embodiment 31

A method for treating cancer in a subject in need thereof, the method comprising administering a therapeutically effective amount of the compound of any one of Embodiments 1-25, or a tautomer, pharmaceutically acceptable salt or prodrug thereof, or the composition of any one of Embodiments 26-28 to the subject.

### Embodiment 32

The method of Embodiment 31, wherein the cancer is an advanced solid tumor resistant to treatment with an immuno-oncology (IO) agent.

### Embodiment 33

The method of Embodiment 32, wherein the cancer is NPM-ALK anaplastic large cell lymphoma.

### Embodiment 34

The method of Embodiment 31, wherein the cancer is an EGFR-mutant non-small cell lung cancer resistant to treatment with a tyrosine kinase inhibitor.

### Embodiment 35

The method of any one of Embodiments 29-34, further comprising administering, or instructing the administration of, a diet low in serine and/or glycine to the subject.

### Embodiment 36

The method of Embodiment 35, wherein the diet is substantially free of serine and/or glycine.

### Embodiment 37

The method of Embodiment 31, wherein the method further comprises administering an RTK inhibitor to the subject.

### Embodiment 38

The method of Embodiment 37, wherein the RTK inhibitor is osimertinib, gefitinib, erlotinib, afatinib, bevacizumab, of trastuzumab.

### Embodiment 39

The method of Embodiment 31, wherein the method further comprises administering a checkpoint inhibitor to the subject.

### Embodiment 40

The method of Embodiment 39, wherein the checkpoint inhibitor is ipilimumab, nivolumab, pembrolizumab, avelumab, or atezolizumab.

### Embodiment 41

The method of any one of Embodiments 39 or 40, wherein the cancer is lymphoma, cancers of the head and neck, lung cancer (e.g., small-cell lung cancer (SCLC) or Non-small cell lung cancer (NSCLC)), bladder cancer, or melanoma (e.g., Merkel cell carcinoma or metastatic melanoma) Embodiment 42. The method of Embodiments 41, wherein the lung cancer is non-small cell lung cancer (NSCLC).

### Embodiment 43

The method of Embodiment 41, wherein the melanoma is Merkel cell carcinoma or metastatic melanoma.

### Embodiment 44

The method of Embodiment 31, wherein the method further comprises administering a ferroptosis inducer to the subject.

### Embodiment 45

The method of Embodiment 44, wherein the ferroptosis inducer is erastin, sorafenib, sulfasalazine, or cisplatin.

### Embodiment 46

The method of any one of Embodiments 44 or 45, wherein the cancer is a ferroptosis-sensitive cancer.

### Embodiment 47

The method of Embodiment 46, wherein the ferroptosis-sensitive cancer is breast cancer, acute myeloid leukemia (AML), pancreatic ductal adenocarcinoma, ovarian cancer, B cell lymphoma, renal cell carcinomas, lung cancer, or glioblastoma.

For purposes of interpreting this specification, the following definitions will apply, and whenever appropriate, terms used in the singular will also include the plural. Terms used in the specification have the following meanings unless the context clearly indicates otherwise.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the present disclosure otherwise claimed.

The term “a,” “an,” “the” and similar terms used in the context of the present disclosure (especially in the context of the claims) are to be construed to cover both the singular and plural unless otherwise indicated herein or clearly contradicted by the context.

As used herein, the terms “alkyl” refers to a hydrocarbon radical of the general formula CₙH₂ₙ₊₁. The alkyl radical may be straight or branched. For example, the term “C₁-C₆ alkyl” refers to a monovalent, straight, or branched aliphatic group containing 1 to 6 carbon atoms (e.g., methyl, ethyl, n-propyl, iso-propyl, n-butyl, iso-butyl, sec-butyl, tert-butyl, n-pentyl, 1-methylbutyl, 2-methylbutyl, 3-methylbutyl, neopentyl, 3,3-dimethylpropyl, hexyl, 2-methylpentyl, and the like). Alkyls are either substituted or unsubstituted (“optionally substituted) The term “heteroaryl” refers to aromatic moieties containing at least one heteroatom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, benzopyranyl, benzothiophenyl, benzoimidazolyl, benzoxazolyl, 1H-benzo[d][1,2,3]triazolyl, and the like.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a hetereoaryl fused to an aryl (e.g., phenyl). Heteroaryls are either substituted or unsubstituted (“optionally substituted).

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may be unsubstituted or substituted with up to three R groups, and at each occurrence R is selected independently from the definition of R.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent.

Combinations of substituents and/or variables are permissible only if such combinations result in stable compounds.

As a person of ordinary skill in the art would be able to understand, for example, a ketone (—CH—C═O) group in a molecule may tautomerize to its enol form (—C═C—OH). Nitrogen-containing compounds may also form tautomers, such as the exemplary tautomers depicted below. Thus, this disclosure is intended to cover all possible tautomers even when a structure depicts only one of them.

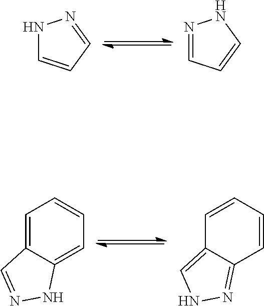 [SMILES: c1cn[nH]c1 ; c1cn[nH]c1 ; c1ccc2[nH]ncc2c1 ; c1ccc2n[nH]cc2c1]

The phrase “pharmaceutically acceptable” indicates that the substance or composition must be compatible chemically and/or toxicologically, with the other ingredients comprising a formulation, and/or the subject being treated therewith.

Unless specified otherwise, the term “compounds of the present disclosure” refers to compounds of structure (I) and subformulae thereof (e.g., structures (Ia), (Ib), (Ic), (Id), (Ie), (If), (Ig), (Ih), (Ii), or (Ij)), as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

It will be recognized by those skilled in the art that the compounds of the present disclosure may contain chiral centers and as such may exist in different isomeric forms. As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Geometric isomers may occur when a compound contains a double bond or some other feature that gives the molecule a certain amount of structural rigidity. If the compound contains a double bond, the substituent may be E or Z configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single a bond.

Unless specified otherwise, the compounds of the present disclosure are meant to include all such possible isomers, including racemic mixtures, optically pure forms and intermediate mixtures. Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. or other equivalent columns, using the appropriate solvent or mixture of solvents to achieve good separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated within the scope of the present disclosure.

As used herein, “pharmaceutically acceptable salts” refer to derivatives of the disclosed compounds wherein the parent compound is modified by making acid or base salts thereof. For example, pharmaceutically acceptable salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate or xinafoate salt form.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table.

In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence the present disclosure further provides co-crystals comprising a compound of the present disclosure.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and idodine, such as ²H, ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of subjects. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of this present disclosure can generally be prepared by conventional techniques known to those skilled in the art or by processes disclosed in the schemes or in the examples and preparations described below (or analogous process to those described herein), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

The term “solvate” means a physical association of a compound of this disclosure with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

As used herein, “polymorph(s)” refer to crystalline form(s) having the same chemical structure/composition but different spatial arrangements of the molecules and/or ions forming the crystals. Compounds of the present disclosure can be provided as amorphous solids or crystalline solids. Lyophilization can be employed to provide the compounds of the present disclosure as a solid.

The term “PKM2 mediated disorder or disease” refers to any disorder or disease which is directly or indirectly regulated by PKM2.

The term “malignancy”, also called cancer, refers to diseases in which abnormal cells divide without control and can invade nearby tissues. Malignant cells can also spread to other parts of the body through the blood and lymph systems. There are several main types of malignancy. Carcinoma is a malignancy that begins in the skin or in tissues that line or cover internal organs. Sarcoma is a malignancy that begins in bone, cartilage, fat, muscle, blood vessels, or other connective or supportive tissue. Leukemia is a malignancy that starts in blood-forming tissue, such as the bone marrow, and causes large numbers of abnormal blood cells to be produced and enter the blood. Lymphoma and multiple myeloma are malignancies that begin in the cells of the immune system. Central nervous system cancers are malignancies that begin in the tissues of the brain and spinal cord.

The term “solid tumor” refers to malignancies/cancers formed of abnormal masses of tissue that usually do not contain cysts or liquid areas. Solid tumors are named/classified according to the tissue/cells of origin. Examples include, but are not limited to, sarcomas and carcinomas.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. Exemplary subjects include humans of any age with risk factors for cancer disease.

As used herein, a subject is “in need of” a treatment if such subject would benefit biologically, medically or in quality of life from such treatment (preferably, a human).

As used herein, the term “inhibit,” “inhibition” or “inhibiting” refers to the reduction or suppression of a given condition, symptom, or disorder, or disease, or a significant decrease in the baseline activity of a biological activity or process.

As used herein, the term “treat,” “treating” or “treatment” of any disease/disorder refers the treatment of the disease/disorder in a subject, particularly in a human, and include: (a) ameliorating the disease/disorder, (i.e., slowing or arresting or reducing the development of the disease/disorder, or at least one of the clinical symptoms thereof); (b) relieving or modulating the disease/disorder, (i.e., causing regression of the disease/disorder), either physically, (e.g., stabilization of a discernible symptom), physiologically, (e.g., stabilization of a physical parameter), or both); (c) alleviating or ameliorating at least one physical parameter including those which may not be discernible by the subject; and/or (d) preventing or delaying the onset or development or progression of the disease or disorder from occurring in a subject, in particular, when such subject is predisposed to the disease or disorder but has not yet been diagnosed as having it.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to (1) at least partially alleviate, inhibit, prevent and/or ameliorate a condition, or a disorder or a disease mediated by PKM2; or (2) modulating the activity of PKM2.

In another non-limiting embodiment, the term “a therapeutically effective amount” refers to the amount of the compound of the present disclosure that, when administered to a cell, or a tissue, or a non-cellular biological material, or a medium, is effective to at least partially modulating the activity of PKM2; or at least partially modulating the expression of PKM2.

The therapeutically effective amount can vary depending on such factors as the size and weight of the subject, the type of illness, or the particular compound of the present disclosure. One of ordinary skill in the art would be able to study the factors contained herein and make the determination regarding the therapeutically effective amount of the compounds of the present disclosure without undue experimentation.

The regimen of administration can affect what constitutes a therapeutically effective amount. The compound of the present disclosure can be administered to the subject either prior to or after the onset of a PKM2 mediated disease or disorder. Further, several divided dosages, as well as staggered dosages, can be administered daily or sequentially, or the dose can be continuously infused, or can be a bolus injection. Further, the dosages of the compound(s) of the present disclosure can be proportionally increased or decreased as indicated by the exigencies of the therapeutic or prophylactic situation.

A cancer that is “resistant” to a particular therapy refers to a cancer that demonstrates persistent disease or complete remission for less than 90 days. In some embodiments, a subject that has a cancer that is resistant to a particular therapy shows no statistically significant objective response to the therapy. A subject is considered to be in “complete remission” if the level of cancerous cells in a sample from the subject is below a detectable or threshold level or if there is no visible tumor on a scan.

### Compounds

As noted above, in one embodiment of the present disclosure, compounds having activity as PKM2 modulators (e.g., activators) are provided, the compounds having the following structure (I):

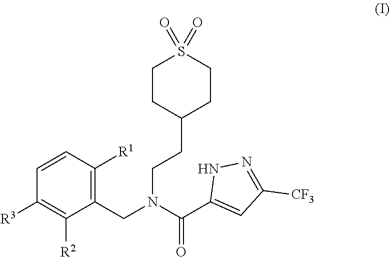

or a pharmaceutically acceptable salt, isotopic form, tautomer or prodrug thereof, wherein:

R¹ is F or NH₂,

R² is H, Cl or Br; or R² joins with R³ to form an optionally substituted heteroaryl; and

R³ is H or optionally substituted C₁-C₆ alkyl; or R³ joins with R² to form an optionally substituted heteroaryl.

In some more specific embodiments, R¹ is F or NH₂, R² is H, Cl or Br, and R³ is H or optionally substituted C₁-C₆ alkyl.

In some embodiments, R³ is H. In some other embodiments, R³ is optionally substituted C₁-C₆ alkyl (e.g., methyl or aminomethyl).

In some embodiments, R² is H. In certain embodiments, embodiments, R² is Cl. In other embodiments, embodiments, R² is Br.

In some specific embodiments, R¹ is F or NH₂, R² is Cl or Br, and R³ is H.

In some embodiments, R² and R³ join to form an optionally substituted heteroaryl. In some more specific embodiments, the heteroaryl is a 5-membered optionally substituted heteroaryl.

In other certain embodiments, the compound is selected from a compound in Table 1 or a pharmaceutically acceptable salt, isotopic form, tautomer, or prodrug thereof.

TABLE 1
Exemplary compounds of structure (I)

| Compound | | |
|---|---|---|
| No. | Compound Structure | Compound Name |
| Ia | 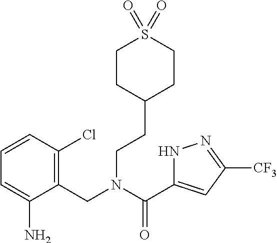 [SMILES: Nc1cccc(Cl)c1CN(CCC1CCS(=O)(=O)CC1)C(=O)c1cc(C(F)(F)F)n[nH]1] | N-(2-amino-6- chlorobenzyl)-N-(2-(1,1- dioxidotetrahydro-2H- thiopyran-4-yl)ethyl)-3- (trifluoromethyl)-1H- pyrazole-5-carboxamide |
| Ib | 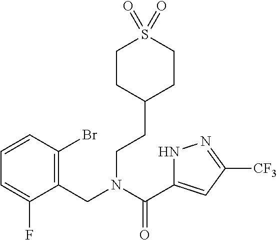 [SMILES: O=C(c1cc(C(F)(F)F)n[nH]1)N(CCC1CCS(=O)(=O)CC1)Cc1c(F)cccc1Br] | N-(2-bromo-6- fluorobenzyl)-N-(2-(1,1- dioxidotetrahydro-2H- thiopyran-4-yl)ethyl)-3- (trifluoromethyl)-1H- pyrazole-5-carboxamide |
| Ic | 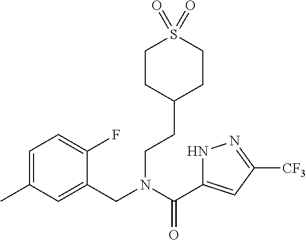 [SMILES: Cc1ccc(F)c(CN(CCC2CCS(=O)(=O)CC2)C(=O)c2cc(C(F)(F)F)n[nH]2)c1] | N-(2-(1,1- dioxidotetrahydro-2H- thiopyran-4-yl)ethyl)-N-(2- fluoro-5-methylbenzyl)-3- (trifluoromethyl)-1H- pyrazole-5-carboxamide |
| Id | 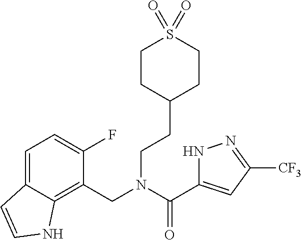 [SMILES: O=C(c1cc(C(F)(F)F)n[nH]1)N(CCC1CCS(=O)(=O)CC1)Cc1c(F)ccc2cc[nH]c12] | N-(2-(1,1- dioxidotetrahydro-2H- thiopyran-4-yl)ethyl)-N- ((6-fluoro-1H-indol-7- yl)methyl)-3- (trifluoromethyl)-1H- pyrazole-5-carboxamide |
| Ie | 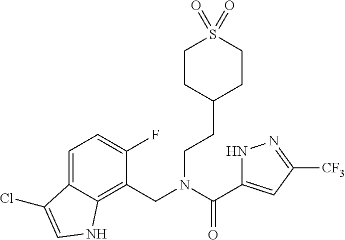 [SMILES: O=C(c1cc(C(F)(F)F)n[nH]1)N(CCC1CCS(=O)(=O)CC1)Cc1c(F)ccc2c(Cl)c[nH]c12] | N-(2-(1,1- dioxidotetrahydro-2H- thiopyran-4-yl)ethyl)-N- ((3-chloro-6-fluoro-1H- indol-7-yl)methyl)-3- (trifluoromethyl)-1H- pyrazole-5-carboxamide |
| If | 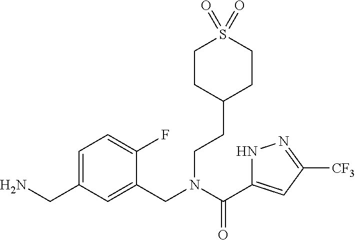 [SMILES: NCc1ccc(F)c(CN(CCC2CCS(=O)(=O)CC2)C(=O)c2cc(C(F)(F)F)n[nH]2)c1] | N-(2-(1,1- dioxidotetrahydro-2H- thiopyran-4-yl)ethyl)-N-(2- fluoro-5- aminomethylbenzyl)-3- (trifluoromethyl)-1H- pyrazole-5-carboxamide |
| Ig | 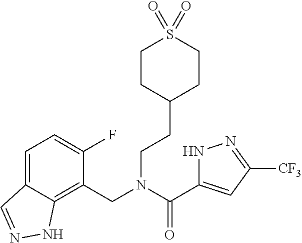 [SMILES: O=C(c1cc(C(F)(F)F)n[nH]1)N(CCC1CCS(=O)(=O)CC1)Cc1c(F)ccc2cn[nH]c12] | N-(2-(1,1- dioxidotetrahydro-2H- thiopyran-4-yl)ethyl)-N- ((6-fluoro-1H-indazol-7- yl)methyl)-3- (trifluoromethyl)-1H- pyrazole-5-carboxamide |
| Ih | 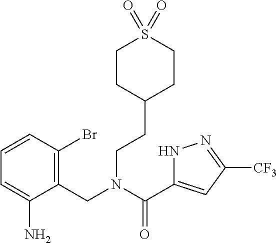 [SMILES: Nc1cccc(Br)c1CN(CCC1CCS(=O)(=O)CC1)C(=O)c1cc(C(F)(F)F)n[nH]1] | N-(2-amino-6- bromobenzyl)-N-(2-(1,1- dioxidotetrahydro-2H- thiopyran-4-yl)ethyl)-3- (trifluoromethyl)-1H- pyrazole-5-carboxamide |
| Ii | 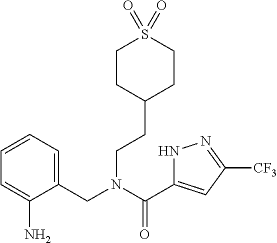 [SMILES: Nc1ccccc1CN(CCC1CCS(=O)(=O)CC1)C(=O)c1cc(C(F)(F)F)n[nH]1] | N-(2-aminobenzyl)-N-(2- (1,1-dioxidotetrahydro-2H- thiopyran-4-yl)ethyl)-3- (trifluoromethyl)-1H- pyrazole-5-carboxamide |
| Ij | 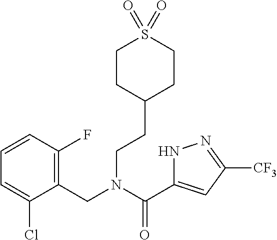 [SMILES: O=C(c1cc(C(F)(F)F)n[nH]1)N(CCC1CCS(=O)(=O)CC1)Cc1c(F)cccc1Cl] | N-(2-chloro-6- fluorobenzyl)-N-(2-(1,1- dioxidotetrahydro-2H- thiopyran-4-yl)ethyl)-3- (trifluoromethyl)-1H- pyrazole-5-carboxamide |

### Pharmaceutical Compositions and Combinations

The compounds of the present disclosure are can also be used in a pharmaceutical composition (e.g., a compound of the present disclosure and at least one pharmaceutically acceptable carrier). A “pharmaceutically acceptable carrier (diluent or excipient)” refers to media generally accepted in the art for the delivery of biologically active agents to subjects, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

In one aspect, the present disclosure provides a pharmaceutical composition comprising a compound of the present disclosure, or a pharmaceutically acceptable salt thereof, and a pharmaceutically acceptable carrier. In a further embodiment, the composition comprises at least two pharmaceutically acceptable carriers, such as those described herein. For purposes of the present disclosure, unless designated otherwise, solvates and hydrates are generally considered compositions. Preferably, pharmaceutically acceptable carriers are sterile. The pharmaceutical composition can be formulated for particular routes of administration such as oral administration, parenteral administration, and rectal administration, etc. In addition, the pharmaceutical compositions of the present disclosure can be made up in a solid form (including capsules, tablets, pills, granules, powders or suppositories), or in a liquid form (including solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of.

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and

e) absorbents, colorants, flavors and sweeteners.

Tablets may be either film coated or enteric coated according to methods known in the art.

Suitable compositions for oral administration include a therapeutically effective amount of a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include a therapeutically effective amount of a compound of the disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present disclosure further provides anhydrous pharmaceutical compositions and dosage forms comprising the compounds of the present disclosure as active ingredients, since water may facilitate the degradation of certain compounds.

Anhydrous pharmaceutical compositions and dosage forms of the disclosure can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The present disclosure further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present disclosure is typically formulated into pharmaceutical dosage forms to provide an easily controllable dosage of the drug and to give the subject an elegant and ergonomic product. The dosage regimen for the compounds of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the subject, and the effect desired. Compounds of this disclosure may be administered in a single daily dose, or the total daily dosage may be administered in divided doses of two, three, or four times daily.

In certain instances, it may be advantageous to administer the compound of the present disclosure in combination with one or more therapeutically active agents independently selected from anti-cancer agents, anti-allergic agents, anti-emetics, pain relievers, immunomodulators and cytoprotective agents.

The term “combination therapy” refers to the administration of two or more therapeutic agents to treat a therapeutic disease, disorder or condition described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. The compound of the present disclosure and additional therapeutic agents can be administered via the same administration route or via different administration routes. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the diseases, conditions or disorders described herein.

General Chemotherapeutic agents considered for use in combination therapies include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), Gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®).

Anti-cancer agents of particular interest for combinations with the compounds of the present disclosure include:

Anti-cancer drugs that increase production of ROS in cancer cells: arsenic trioxide (As₂O₃), 2-methoxyestradiol, L-asparaginase, G202, nelfinavir, PARP inhibitors, erastin, lanperasone, camptothecin, inostamycin, Adriamycin, doxorubicin, daunorubicin, epirubicin, idarubicin, nemorubicin, sabarubicin, valrubicin, mitoxantrone, pixantrone, bortezomib, N-benzyloxycarbonyl-Ile-Glu(O-tert-butyl)-Ala-leucinal (PSI), sorafenib, erlotinib, retaspimycin hydrochloride (IPI-504), 17-allylamino-17-demethoxygeldanamycin (17-AAG), paclitaxel, docetaxel, vincristine, vinblastine, vindesine, anti-folates, cisplatin, carboplatin, oxaliplatin, N-(4-hydroxyphenyl) retinamide, NOV-002, Sulphasalazine, 6-anicotinamide, dibenzophenanthridine, Buthionine sulphoximine, valdecoxib, paracoxib, rofecoxib, Cox 189, AGX-891, AG-221, and compounds disclosed in, e.g., U.S. Publication Numbers 2015/0197498, 2011/0053938, 2012/0259004 and PCT Publication Number WO 2012/123076, the full disclosures of which are herein incorporated by reference in their entirety.

Kinase inhibitors: tyrosine kinase inhibitors, cyclin dependent kinase inhibitors, mitogen-activated protein kinase inhibitors, and phosphoinositide 3-kinase inhibitors.

Purine antimetabolites and/or inhibitors of de novo purine synthesis: pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®).

MTAP inhibitors: (3R,4S)-1-((4-amino-5H-pyrrolo[3,2-d]pyrimidin-7-yl)methyl)-4-((methylthio)methyl)pyrrolidin-3-ol (MT-DADMe-Immucillin-A, CAS 653592-04-2).

Methylthioadenosine: ((2R,3R,4S,5S)-2-(6-amino-9H-purin-9-yl)-5-((methylthio)methyl)tetrahydrofuran-3,4-diol, CAS 2457-80-9).

Epidermal growth factor receptor (EGFR) inhibitors: Erlotinib hydrochloride (Tarceva®), Gefitnib (Iressa®), ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.). Additional EGFR inhibitors are described in, for example, WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), which are incorporated by reference herein.

EGFR antibodies: Cetuximab (Erbitux®), C225, and anti-EGFR 22Mab (ImClone Systems, Inc., New York, N.Y.)

MET inhibitors: Capmatinib (INC280, CAS 1029712-80-8).

Platelet-derived Growth Factor (PDGF) receptor inhibitors: Imatinib (Gleevec®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Quizartinib (AC220, CAS 950769-58-1); Pazopanib (Votrient®); Axitinib (Inlyta®); Sorafenib (Nexavar®); Vargatef (BIBF1120, CAS 928326-83-4); Telatinib (BAY57-9352, CAS 332012-40-5); Vatalanib dihydrochloride (PTK787, CAS 212141-51-0); and Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Phosphoinositide 3-kinase (PI3K) inhibitors: 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 4-(trifluoromethyl)-5-(2,6-dimorpholinopyrimidin-4-yl)pyridin-2-amine (also known as BKM120 or NVP-BKM120, and described in PCT Publication No. WO2007/084786); Alpelisib (BYL719): (5Z)-5-[[4-(4-Pyridinyl)-6-quinolinyl]methylene]-2,4-thiazolidinedione (GSK1059615, CAS 958852-01-2); 5-[8-methyl-9-(1-methylethyl)-2-(4-morpholinyl)-9H-purin-6-yl]-2-pyrimidinamine (VS-5584, CAS 1246560-33-7) and everolimus (AFINITOR*).

Cyclin-Dependent Kinase (CDK) inhibitors: Ribociclib (LEE011, CAS 1211441-98-3); Aloisine A; Alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); Crizotinib (PF-02341066, CAS 877399-52-5); 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); 1-Methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265, CAS 927880-90-8); Indisulam (E7070); Roscovitine (CYC202); 6-Acetyl-8-cyclopentyl-5-methyl-2-(5-piperazin-1-yl-pyridin-2-ylamino)-8H-pyrido[2,3-d]pyrimidin-7-one, hydrochloride (PD0332991); Dinaciclib (SCH727965); N-[5-[[(5-tert-Butyloxazol-2-yl)methyl]thio]thiazol-2-yl]piperidine-4-carboxamide (BMS 387032, CAS 345627-80-7); 4-[[9-Chloro-7-(2,6-difluorophenyl)-5H-pyrimido[5,4-d][2]benzazepin-2-yl]amino]-benzoic acid (MLN8054, CAS 869363-13-3); 5-[3-(4,6-Difluoro-1H-benzimidazol-2-yl)-1H-indazol-5-yl]-N-ethyl-4-methyl-3-pyridinemethanamine (AG-024322, CAS 837364-57-5); 4-(2,6-Dichlorobenzoylamino)-1H-pyrazole-3-carboxylic acid N-(piperidin-4-yl)amide (AT7519, CAS 844442-38-2); 4-[2-Methyl-1-(1-methylethyl)-1H-imidazol-5-yl]-N-[4-(methylsulfonyl)phenyl]-2-pyrimidinamine (AZD5438,CAS 602306-29-6); Palbociclib (PD-0332991); and (2R,3R)-3-[[2-[[3-[[S(R)]-S-cyclopropylsulfonimidoyl]-phenyl]amino]-5-(trifluoromethyl)-4-pyrimidinyl]oxy]-2-butanol (BAY 10000394).

p53-MDM2 inhibitors: (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone (RG7112), 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid (RG7388), SAR299155, 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid (AMG232), {(3R,5R,6S)-5-(3-Chlorophenyl)-6-(4-chlorophenyl)-1-[(2S,3S)-2-hydroxy-3-pentanyl]-3-methyl-2-oxo-3-piperidinyl}acetic acid (AM-8553), (+)-4-[4,5-Bis(4-chlorophenyl)-2-(2-isopropoxy-4-methoxy-phenyl)-4,5-dihydro-imidazole-1-carbonyl]-piperazin-2-one (Nutlin-3), 2-Methyl-7-[Phenyl(phenylamino)methyl]-8-quinolinol (NSC 66811), 1-N-[2-(1H-indol-3-yl)ethyl]-4-N-pyridin-4-ylbenzene-1,4-diamine (JNJ-26854165), 4-[4,5-bis(3,4-chlorophenyl)-2-(2-isopropoxy-4-methoxy-phenyl)-4,5-dihydro-imidazole-1-carboxyl]-piperazin-2-one (Caylin-1), 4-[4,5-bis(4-trifluoromethyl-phenyl)-2-(2-isopropoxy-4-methoxy-phenyl)-4,5-dihydro-imidazole-1-carboxyl]-piperazin-2-one (Caylin-2), 5-[[3-Dimethylamino)propyl]amino]-3,10-dimethylpyrimido[4,5-b]quinoline-2,4(3H,10H)-dione dihydrochloride (HLI373) and trans-4-Iodo-4′-boranyl-chalcone (SC204072).

Mitogen-activated protein kinase (MEK) inhibitors: XL-518 (also known as GDC-0973, CAS No. 1029872-29-4, available from ACC Corp.); Selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO 2003/077914); 2-[(2-Chloro-4-iodophenyl)amino]-N-(cyclopropylmethoxy)-3,4-difluoro-benzamide (also known as CI-1040 or PD184352 and described in PCT Publication No. WO 2000/035436); N-[(2R)-2,3-Dihydroxypropoxy]-3,4-difluoro-2-[(2-fluoro-4-iodophenyl)amino]-benzamide (also known as PD0325901 and described in PCT Publication No. WO 2002/006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO 2007/014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO 2003/076424); 2′-Amino-3′-methoxyflavone (also known as PD98059 available from Biaffin GmbH & Co., KG, Germany); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS No. 1204531-26-9); Trametinib dimethyl sulfoxide (GSK-1120212, CAS No. 1204531-25-80); 2-(2-Fluoro-4-iodophenylamino)-N-(2-hydroxyethoxy)-1,5-dimethyl-6-oxo-1,6-dihydropyridine-3-carboxamide (AZD 8330); 3,4-Difluoro-2-[(2-fluoro-4-iodophenyl) amino]-N-(2-hydroxyethoxy)-5-[(3-oxo-[1,2]oxazinan-2-yl)methyl]benzamide (CH 4987655 or Ro 4987655); and 5-[(4-Bromo-2-fluorophenyl)amino]-4-fluoro-N-(2-hydroxyethoxy)-1-methyl-1H-Benzimidazole-6-carboxamide (MEK162).

B-RAF inhibitors: Regorafenib (BAY73-4506, CAS 755037-03-7); Tuvizanib (AV951, CAS 475108-18-0); Vemurafenib (Zelboraf®, PLX-4032, CAS 918504-65-1); Encorafenib (also known as LGX818); 1-Methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS No. 927880-90-8); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662), dabrafenib (Tafinlar®), and N-(3-(5-chloro-1H-pyrrolo[2,3-b]pyridine-3-carbonyl)-2,4-difluorophenyl)propane-1-sulfonamide (also known as PLX4720).

ALK inhibitors: Crizotinib (Xalkori®).

Anthracyclines: doxorubicin, daunorubicin, epirubicin, idarubicin, nemorubicin, sabarubicin, and valrubicin.

Anthracenediones: mitoxantrone and pixantrone.

Proteasome inhibitors: bortezomib and N-benzyloxycarbonyl-Ile-Glu(O-tert-butyl)-Ala-leucinal (PSI).

Tyrosine kinase inhibitors: sorafenib and erlotinib.

HSP90 inhibitors: retaspimycin hydrochloride (IPI-504) and 17-allylamino-17-demethoxygeldanamycin (17-AAG).

Taxanes: paclitaxel and docetaxel.

Vinca alkaloids: vincristine, vinblastine, and vindesine.

Platinum coordinating complexes: cisplatin, carboplatin, and oxaliplatin.

Retinoid derivatives: N-(4-hydroxyphenyl) retinamide.

Glutathione disulfide mimetics: NOV-002.

Inhibitors of systine/glutamate transporter XCT: Sulphasalazine.

Inhibitors of glucose-6-phosphate dehydrogenase: 6-anicotinamide.

Glutaminase inhibitors: dibenzophenanthridine.

Glutamate-cysteine ligase complex inhibitors: Buthionine sulphoximine.

COX2 inhibitors: Vioxx, CELEBREX (celecoxib), valdecoxib, paracoxib, rofecoxib, and Cox 189.

Mutant IDH1 and IDH2 isoform inhibitors: AGX-891 and AG-221.

Pyrimidine analogs: UFT, capecitabine, gemcitabine and cytarabine.

Purine analogs: mercaptopurine and thioguanine.

Alkyl sulfonates: busulfan, improsulfan and piposulfan.

Aziridines: benzodepa, carboquone, meturedepa and uredepa.

Matrix metalloproteinase (MMP) inhibitors: AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds. Other MMP inhibitors include those described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated by reference herein.

VEGF inhibitors: SU-5416, SU-6668 (Sugen Inc., South San Francisco, Calif.), IM862 (Cytran Inc., Kirkland, Wash.), anti-VEGF monoclonal antibody of Genentech, Inc., angiozyme (a synthetic ribozyme from Ribozyme (Boulder, Colo.) and Chiron (Emeryville, Calif.)), and the VEGF inhibitors described in WO 01/60814 A3 (published Aug. 23, 2001), WO 99/24440 (published May 20, 1999), PCT International Application PCT/IB99/00797 (filed May 3, 1999), WO 95/21613 (published Aug. 17, 1995), WO 99/61422 (published Dec. 2, 1999), U.S. Pat. No. 5,834,504 (issued Nov. 10, 1998), WO 01/60814, WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference.

pErbB2 receptor inhibitors: GW-282974 (Glaxo Wellcome plc), AR-209 (Aronex Pharmaceuticals Inc., The Woodlands, Tex.), 2B-1 (Chiron), and the inhibitors described in in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are incorporated in its entirety herein by reference.

ErbB2 receptor inhibitors: Herceptin, and the inhibitors described in U.S. Pat. No. 6,284,764 (issued Sep. 4, 2001), which is incorporated in its entirety herein by reference.

Topoisomerase inhibitors: CAMPTOSAR (irinotecan).

Anti-androgens: substituted ureas such as hydroxyurea.

Methylhydrazine derivatives: procarbazine.

Adrenocortical suppressants: mitotane and aminoglutethimide.

Hormone and hormone antagonists: adrenocorticosteriods (e.g., prednisone), progestins (e.g., hydroxyprogesterone caproate), estrogens (e.g., diethylstilbesterol), antiestrogens (e.g., tamoxifen), androgens (e.g., testosterone propionate), and aromatase inhibitors (e.g., anastrozole, and AROMASIN (exemestane)).

Alkylating agents: fluorouracil (5-FU).

Ethyleneimines and methylmelamines: altretamine, triethylenemelamine, triethylenephosphoramide, triethylenethiophosphoramide and trimethylolmelamine.

Nitrogen mustards: chlorambucil, cyclophosphamide, estramustine, ifosfamide, novembrichin, prednimustine and uracil mustard.

Triazines: dacarbazine.

Folic acid analogs: methotrexate and pteropterin.

Epipodophyllotoxins: etoposide and teniposide.

Antibiotic chemotherapeutic agents: daunorubicin, doxorubicin, epirubicin, mitomycin, dactinomycin, temozolomide, plicamycin, and bleomycin.

Enzymatic chemotherapeutic agents: L-asparaginase.

In further embodiments, the anti-cancer agent is a mitotic inhibitor, a cell cycle inhibitor, an enzyme, a biological response modifier, an anti-hormone, an anti-metabolites (e.g., an anti-folate), or a combination thereof.

Additionally, combinations of the PKM2 modulating compounds disclosed herein (e.g., compounds of structure (I)) with additional therapies are specifically contemplated. The compounds of structure (I) or a pharmaceutically acceptable salt, isotopic form, tautomer or prodrug thereof may be combined with, for example, therapies involving a low serine diet, a kinase inhibitor, a checkpoint inhibitor, or a compound that induces ferroptosis.

Serine metabolism may be altered in diseased cells, including tumor cells. Restriction of serine may affect cellular metabolic and proliferation processes, such as by limiting nucleotide and protein biosynthesis, or by reducing the production of glycine. Therapies that modulate serine metabolism, including administering therapeutic agents that restrict cellular serine production or otherwise lower cellular serine levels, may be combined with compounds of structure (I) or a pharmaceutically acceptable salt, isotopic form, tautomer or prodrug thereof to treat disease (e.g., cancer).

Kinase inhibitors disrupt signal transduction between cells and include inhibitors of members of the receptor tyrosine kinase (RTK) family. The RTK family includes EGFR, which may be inhibited by osimertinib, gefitinib, erlotinib, and afatinib; VEGF, which may be inhibited by bevacizumab; and ErbB2, which may be inhibited by trastuzumab. Therapeutic agents that inhibit members of the RTK family, including therapeutic agents that inhibit EGFR, VEGF, or ErbB2, may be administered in combination with compounds of structure (I) or a pharmaceutically acceptable salt, isotopic form, tautomer or prodrug thereof to treat disease (e.g., cancer).

Immunological checkpoints prevent the immune system from attacking cells in an indiscriminate manner and can hinder T cells from killing diseased cells that have avoided immune attack. Inhibiting checkpoint proteins may be used to initiate or boost the immune response against such cells. Checkpoint inhibitors include inhibitors of CTLA-4, PD-1 and PD-L1, such as ipilimumab, nivolumab, pembrolizumab, avelumab, and atezolizumab. Therapies that inhibit checkpoint proteins, including therapeutic agents that inhibit CTLA-4, PD-1 or PD-L1, may be administered in combination with a compound of structure (I) or a pharmaceutically acceptable salt, isotopic form, tautomer or prodrug thereof to treat disease.

Ferroptosis is an iron-dependent type of programmed cell death that involves disruption of the oxidative degradation of lipids due, in part, to reduced activity of glutathione-dependent antioxidation enzymes. Ferroptotic cells accumulate lipid peroxides and may exhibit higher cellular concentrations of reactive oxygen species (ROS) than normal cells. Inducing ferroptosis in cells can occur by multiple pathways, including by reducing the levels of cellular glutathione, leading to inhibition of tumor growth and enhanced sensitivity to additional therapies, such as doxorubicin.

Inducers of ferroptosis include erastin, sorafenib, sulfasalazine, and cisplatin. Therapies that induce ferroptosis, including therapeutic agents that reduce cellular glutathione levels and/or increase cellular levels of ROS, may be administered in combination with a compound of structure (I) or a pharmaceutically acceptable salt, isotopic form, tautomer or prodrug thereof to treat disease.

In embodiments, a compound of structure (I) or a pharmaceutically acceptable salt, isotopic form, tautomer or prodrug thereof is combined with a low serine diet. In some embodiments, a compound of structure (I) or a pharmaceutically acceptable salt, isotopic form, tautomer or prodrug thereof is combined with a kinase inhibitor. In some embodiments, a compound of structure (I) or a pharmaceutically acceptable salt, isotopic form, tautomer or prodrug thereof is combined with a checkpoint inhibitor. In some embodiments, a compound of structure (I) or a pharmaceutically acceptable salt, isotopic form, tautomer or prodrug thereof is combined with a compound that induces ferroptosis.

In some embodiments, a compound of structure (I) or a pharmaceutically acceptable salt, isotopic form, tautomer or prodrug thereof is combined with an anthracycline. In some embodiments, a compound of structure (I) or a pharmaceutically acceptable salt, isotopic form, tautomer or prodrug thereof is combined with an anthracenedione. In some embodiments, a compound of structure (I) or a pharmaceutically acceptable salt, isotopic form, tautomer or prodrug thereof is combined with a proteasome inhibitor.

In further embodiments, a compound of structure (I) or a pharmaceutically acceptable salt, isotopic form, tautomer or prodrug thereof is combined with an immunomodulator. Immunomodulators of particular interest for combinations with the compounds of the present disclosure include: Afutuzumab (available from Roche®); Pegfilgrastim (Neulasta®); Lenalidomide (CC-5013, Revlimid®); Thalidomide (Thalomid®), Actimid (CC4047); and IRX-2 (mixture of human cytokines including interleukin 1, interleukin 2, and interferon γ, CAS No. 951209-71-5, available from IRX Therapeutics).

Some subjects may experience allergic reactions to the compounds of the present disclosure and/or other anti-cancer agent(s) during or after administration; therefore, anti-allergic agents are often administered to minimize the risk of an allergic reaction. Suitable anti-allergic agents include corticosteroids (Knutson, S., et al., PLoS One, DOI:10.1371/journal.pone.0111840 (2014)), such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some subjects may experience nausea during and after administration of the compound of the present disclosure and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the subject more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g., Patents International (e.g., IMS World Publications).

In one embodiment, the present disclosure provides pharmaceutical compositions comprising at least one compound of the present disclosure or a pharmaceutically acceptable salt thereof together with a pharmaceutically acceptable carrier suitable for administration to a human or animal subject, either alone or together with other anti-cancer agents.

In another embodiment, the present disclosure provides methods of treating human or animal subjects suffering from a cellular proliferative disease, such as malignancy. The present disclosure provides methods of treating a human or animal subject in need of such treatment, comprising administering to the subject a therapeutically effective amount of a compound of the present disclosure or a pharmaceutically acceptable salt thereof, either alone or in combination with other anti-cancer agents.

In particular, compositions will either be formulated together as a combination therapeutic or administered separately.

In combination therapy for treatment of a malignancy, the compound of the present disclosure and other anti-cancer agent(s) may be administered simultaneously, concurrently or sequentially with no specific time limits, wherein such administration provides therapeutically effective levels of the two compounds in the body of the subject.

In a preferred embodiment, the compound of the present disclosure and the other anti-cancer agent(s) is generally administered sequentially in any order by infusion or orally. The dosing regimen may vary depending upon the stage of the disease, physical fitness of the subject, safety profiles of the individual drugs, and tolerance of the individual drugs, as well as other criteria well-known to the attending physician and medical practitioner(s) administering the combination. The compound of the present disclosure and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present disclosure, a kit comprising two or more separate pharmaceutical compositions, at least one of which contains a compound of the present disclosure is provided. In one embodiment, the kit comprises means for separately retaining said compositions, such as a container, divided bottle, or divided foil packet. An example of such a kit is a blister pack, as typically used for the packaging of tablets, capsules and the like.

The kit of the present disclosure may be used for administering different dosage forms, for example, oral and parenteral, for administering the separate compositions at different dosage intervals, or for titrating the separate compositions against one another. To assist compliance, the kit of the present disclosure typically comprises directions for administration.

A compound of the present disclosure may also be used to advantage in combination with known therapeutic processes, for example, the administration of hormones or especially radiation. A compound of the present disclosure may in particular be used as a radiosensitizer, especially for the treatment of tumors which exhibit poor sensitivity to radiotherapy.

In the combination therapies of the present disclosure, the compound of the present disclosure and the other therapeutic agent may be manufactured and/or formulated by the same or different manufacturers. Moreover, the compound of the present disclosure and the other therapeutic (or pharmaceutical agent) may be brought together into a combination therapy: (i) prior to release of the combination product to physicians (e.g. in the case of a kit comprising the compound of the present disclosure and the other therapeutic agent); (ii) by the physician themselves (or under the guidance of the physician) shortly before administration; (iii) in the subject themselves, e.g. during sequential administration of the compound of the present disclosure and the other therapeutic agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition or combination of the present disclosure can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the therapeutically effective amount of each of the active ingredients necessary to prevent, treat or inhibit the progress of the disorder or disease.

The above-cited dosage properties may be demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

### Pharmacology and Utility

The compounds and compositions of the present disclosure are effective agents for treating various malignancies, including cancers. In particular, experimental evidence set forth herein shows that tumor growth can be reduced by administering compounds of structure (I). In particular, xenograft models illustrate the present compounds are effective for reducing tumor growth in A549 cells. The present compounds are active as PKM2 modulators (e.g., activators) and are useful for methods of accomplishing the same.

Accordingly, one embodiment provides a method for treating a disease by modulating pyruvate kinase muscle isozyme M2 (PKM2) activity in a subject in need thereof, the method comprising administering a therapeutically effective amount of a compound as or composition as described in any of the foregoing embodiments to the subject. In more specific embodiments, the modulating comprises activating PKM2. For example, the activating of PKM2 may be for treatment of cancer.

In various other embodiments, the disclosure is directed to a method for treating cancer comprising administering any of the above described compounds of structure (I) or compositions thereof to a subject (e.g., mammal) in need thereof. In some specific embodiments, the cancer is lung cancer (e.g., non-small cell lung cancer). For example, in certain embodiments the cancer is an EGFR-mutant non-small cell lung cancer, optionally which is resistant to treatment with tyrosine kinase inhibitors (TKI).

In some embodiments, the cancer is a solid tumor, for example an advanced solid tumor, optionally which is resistant to treatment with an immuno-oncology (IO) agent. In some other embodiments, the cancer is lymphoma, for example large cell lymphoma such as NPM-ALK anaplastic large cell lymphoma.

In other embodiments, the compounds of the present disclosure inhibit cancer cell proliferation. The compounds and compositions of the disclosure will also find utility in a broad range of diseases, disorders, and conditions mediated by PKM2.

Such diseases may include by way of example and not limitation, cancers such as lung cancer, NSCLC (non-small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy.

Therefore, in one embodiment, the compounds of the present disclosure may be useful to treat any one of the above listed malignancy types which are characterized by PKM2-deficiency.

Selective modulation of PKM2 in PKM2-deficient disease (e.g., malignancy) may provide certain therapeutic benefits. Therefore, compounds of the present disclosure have favorable therapeutic benefits for PKM2 mediated disorder or disease as a result of their selective modulation on PKM2-deficient cells.

Additionally, a combination of a PKM2 modulating compound as disclosed herein (i.e., a compound of structure (I)) with therapies involving a low serine diet, a kinase inhibitor, a checkpoint inhibitor, a ferroptosis inducer, or a reactive oxygen species, may be useful for treating a variety of cancers, including but not limited to pancreatic cancer, colorectal cancer, leukemias and lymphomas including CML, esophageal cancer, glioma, gastrointestinal cancer including GIST, renal cell carcinoma, breast cancer, cancers of the head and neck, lung cancer including NSCLC, bladder cancer, and melanomas (e.g., Merkel cell melanoma or metastatic melanoma).

For example, one embodiment provides a method for treating cancer in a subject in need thereof, the method comprising administering a therapeutically effective amount of a compound of structure (I) or a tautomer, pharmaceutically acceptable salt or prodrug thereof and an RTK inhibitor (e.g., osimertinib, gefitinib, erlotinib, afatinib, bevacizumab, of trastuzumab). In some more specific embodiments, the cancer is CML, colorectal cancer, esophageal cancer, glioma, gastrointestinal cancer (e.g., GIST), renal cell carcinoma, breast cancer, cancers of the head and neck, or lung cancer.

Another embodiment provides a method for treating cancer in a subject in need thereof, the method comprising administering a therapeutically effective amount of a compound of structure (I) or a tautomer, pharmaceutically acceptable salt or prodrug thereof and a checkpoint inhibitor (e.g., ipilimumab, nivolumab, pembrolizumab, avelumab, or atezolizumab). In some embodiments, the cancer is lymphoma, cancers of the head and neck, lung cancer (e.g., NSCLC), bladder cancer, or melanoma (e.g., Merkel cell carcinoma or metastatic melanoma).

Yet another embodiment provides method for treating cancer in a subject in need thereof, the method comprising administering a therapeutically effective amount of a compound of structure (I) or a tautomer, pharmaceutically acceptable salt or prodrug thereof and a ferroptosis inducer (e.g., erastin, sorafenib, sulfasalazine, or cisplatin). In some embodiments, the cancer is a ferroptosis-sensitive cancer. For example, in some more specific embodiments, the ferroptosis-sensitive cancer is breast cancer, AML, pancreatic ductal adenocarcinoma, ovarian cancer, B cell lymphoma, renal cell carcinomas, lung cancer, or glioblastoma. The compounds of present disclosure in free form or in pharmaceutically acceptable salt form, exhibit valuable pharmacological properties, which can be demonstrated at least by using any one of the following test procedures. Compounds of the present disclosure were assessed for their ability to modulate PKM2 activity in biochemical assays and cellular assays.

In various different embodiments, the methods further comprise administering, or instructing the administration of, a diet low in serine and/or glycine to the subject. For example, in some embodiment the diet is substantially free or free of serine and/or glycine.

Certain embodiments of the disclosed methods further include administering a chemotherapeutic agent concurrently, prior to or after administering the compound of structure (I).The following examples are provided for purposes of illustration, not limitation.

### EXAMPLES

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd}-ed., Wiley-VCH Weinheim, Germany (1999), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

Additionally, the compounds of present disclosure exhibit valuable pharmacological properties, which can be demonstrated at least by using any one of the following test procedures. Accordingly, compounds of the present disclosure were assessed in biochemical assays.

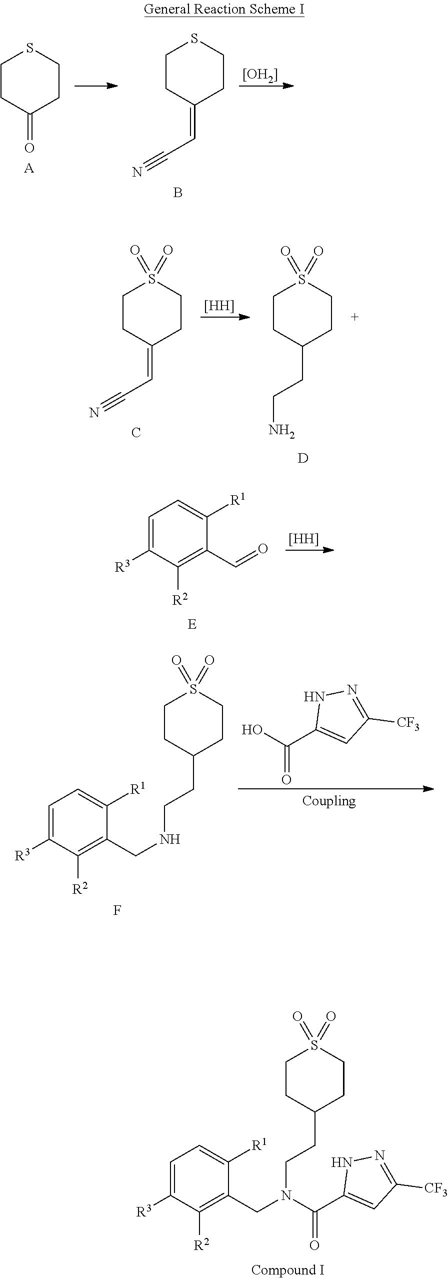

As shown in General Reaction Scheme 1, compound A is prepared according to known methods or purchased as a commercial reagent. Referring to General Reaction Scheme I, A can be reacted with an appropriate reagent (e.g., diethyl (cyanomethyl)phosphonate) to introduce a cyano containing substituent to afford B. B can then be oxidized under suitable conditions (e.g., o-zone in methanol/water) to C and then reduced (e.g., using Raney nickel and hydrogen gas) to yield D. D is further reacted with E under appropriate reductive amination conditions (e.g., sodium borohydride) to provide F. As a final step, F is reacted under amino acid coupling conditions (e.g., HATU, DIPEA) to afford the desired product, a compound of structure I. It should be noted that, as an alternative, R¹, R², and R³ may be selected or modified during any step of the reaction sequence based on over compatibility with the overall reaction scheme and desired reaction selectivity (e.g., conversion of —NO₂ to —NH₂ using Fe/NH₄Cl/ethanol).

### Example 1

### Pharmacokinetic Profiling

Compounds were pre-incubated with 2 nM PKM2 in reaction buffer (50 mM Tris-HCl, pH 8.0, 200 mM KCl, 30 mM MgCl₂, 2 mM Dithiothreitol (DTT), 5% Dimethyl sulfoxide (DMSO)) for 30 min at ambient temperature. Adenosine diphosphate (ADP) and Phosphoenolpyruvic acid (PEP) were then added to final concentrations of 75 μM and 15 μM, respectively. After 30 min, Adenosine triphosphate (ATP) formation was measured by Kinase Glo (Promega, Madison, Wis.), and AC₅₀ values were determined using Prism (GraphPad Software, Inc., La Jolla, Calif.).

A549 cells were seeded at 5000 cells per well (in 96-well plate) in Basal Medium Eagle (BME) media lacking nonessential amino acids+5% dialyzed serum. After 18 hours, a negative control (i.e., DMSO only) or a representative compound in 0.1% final concentration DMSO was added. After 72 hours, cell viability was determined by ATPlite assay and EC₅₀ values were determined using Prism GraphPad Software (La Jolla, Calif.).

TABLE 2
PKM2 AC₅₀ and A549 EC₅₀ determined for
Compounds Ia, lb, Ic, Id, Ie, If, Ig, Ih, Ii, and Ij

| Compound | PKM2 AC₅₀(nM)¹ | A549 EC₅₀ (nM)² |
|---|---|---|
| Ia | *** | +++ |
| Ib | ** | + |
| Ic | ** | ++ |
| Id | *** | +++ |
| Ie | *** | + |
| If | ** | + |
| Ig | ** | + |
| Ih | * | + |
| Ii | * | ++ |
| Ij | * | Not Tested |

¹* = greater than 50 nM;
** = 15-50 nM;
*** less than 15 nM
²+ = greater than 100 nM;
++ 50-100 nM;
+++ less than 50 nM

### Example 2

### Synthesis of Compound Ia 4-(2-((2-chloro-6-nitrobenzyl) amino) ethyl) tetrahydro-2H-thiopyran-1,1-dioxide (4c)

To a stirred solution of 4-(2-aminoethyl)tetrahydro-2H-thiopyran-1,1-dioxide (4b; 5.7 g, 0.0321 mol, 1.1 eq.) in ethanol (2.0 mL), 2-chloro-6-nitrobenzaldehyde (4a; 5.42 g, 0.0292 mol, 1.0 eq) was added at room temperature and the reaction mixture was stirred for 16h at room temperature. After confirming the consumption of starting aldehyde by TLC, the reaction mixture was cooled to 5° C.-10° C., and sodium borohydride (3.0 eq) was added to it portion wise. After completing the addition, the reaction temperature brought to room temperature and stirring was continued for additional 3h. After confirming the completion of reaction by TLC, reaction mixture was quenched with water. Ethanol was evaporated completely under reduced pressure and aqueous layer was extracted with ethyl acetate (200 mL). Combined organic layer was washed with brine solution (100 mL), dried over sodium sulfate and evaporated under reduced pressure. Crude compound obtained was purified by flash column chromatography using silica gel to afford 4-(2-((2-chloro-6-nitrobenzyl)amino)ethyl)tetrahydro-2H-thiopyran-1,1-dioxide (4c) as a light brown solid (5.38 g, 53.3%.). LCMS (ELSD) results are shown in Table 3.

### N-(2-chloro-6-nitrobenzyl)-N-(2-(1,1-dioxidotetrahydro-2H-thiopyran-4-yl) ethyl-3-(trifluoromethyl)-1H-pyrazole-5-carboxamide (4d)

To a stirred solution of 4-(2-((2-chloro-6-nitrobenzyl)amino)ethyl) tetrahydro-2H-thiopyran-1,1-dioxide (4c; 5.0 g, 0.0144 mol, 1.0 eq.) in DMF (100 mL) at 10° C.-15° C., 3-(trifluoromethyl)-1H-pyrazole-5-carboxylic acid (2.60 g, 0.0144 mol, 1.0 eq), HATU (8.21 g, 0.0216, 1.5 eq) and N,N-diisopropylethylamine (3.72 g, 0.0288 mol, 2.0 eq) were added. The reaction mixture was allowed to reach room temperature and then stirred for 16 hours. After confirming the completion of reaction by TLC, the reaction mixture was diluted with water and extracted with ethyl acetate (10 mL). The organic layer was washed with water (10 mL×3), dried over sodium sulfate and the solvent was evaporated under reduced pressure. The crude compound obtained was purified by flash column chromatography to afford N-(2-chloro-6-nitrobenzyl)-N-(2-(1,1-dioxidotetrahydro-2H-thiopyran-4-yl)ethyl-3-(trifluoromethyl)-1H-pyazole-5-carboxamide (4d) as an off-white solid (4.2 g, 57.4%.). LCMS (ELSD) results are shown in Table 3.

### N-(2-amino-6-chlorobenzyl)-N-(2-(1,1-dioxidotetrahydro-2H-thiopyran-4-yl) ethyl-3-(trifluoromethyl)-1H-pyazole-5-carboxamide (Compound Ia)

To a stirred solution of 4-(2-((2-chloro-6-nitrobenzyl)amino)ethyl)tetrahydro-2H-thiopyran-1,1-dioxide (4d; 4.0 g, 0.0079 mol, 1.0 eq.) in ethanol (40 mL) at room temperature, aqueous solution of ammonium chloride (2.50 g in 5.0 mL water, 0.0472 mol, 6.0 eq) and iron powder (2.64 g, 0.0472 mol, 6.0 eq) were added. Next, the reaction mixture was heated to 85° C. for 3h. After confirming the completion of reaction by TLC, the reaction mixture was cooled to room temperature and filtered through a celite bed. The celite bed was thoroughly washed with ethyl acetate (20 mL). The filtrate was washed well with water. The combined organic layers was dried over sodium sulfate and evaporated under reduced pressure. The crude compound obtained was purified by flash column chromatography to afford N-(2-amino-6-chlorobenzyl)-N-(2-(1,1-dioxidotetrahydro-2H-thiopyran-4-yl)-ethyl-3-(trifluoromethyl)-1H-pyazole-5-carboxamide (Compound Ia) as off-white solid (2.0 g, 53.2%.). LCMS (ELSD), HPLC, and ¹H NMR results are shown in Table 4.

### Example 3

### Synthesis of Compound IB 2-(tetrahydro-4H-thiopyran-4-ylidine) acetonitrile (5b)

To an ice cold stirred solution of diethyl cyanomethylphosphonate (109.7 g, 0.6197 mol, 1.2 eq.) in dry THE (600 mL), sodium hydride (60% in mineral oil, 32.0 g, 0.672 mol, 1.3 eq) was added, portion wise. The reaction mixture was stirred for 15 minutes and then tetrahydro-4H-thiopyran-4-one (60.0 g, 0.5164 mol, 1.0 eq) was added slowly as a solution in dry THE (120 mL, 2.0 volumes). Stirring was continued for another 2h at the same temperature (0° C.-5° C.). After confirming the completion of reaction by TLC, the reaction was quenched by the slow addition of ice cold water. Then reaction mixture was extracted with DCM (600 mL), the organic layer was dried over sodium sulfate and the solvent was evaporated under reduced pressure. The crude compound was purified by column chromatography to afford 2-(tetrahydro-4H-thiopyran-4-ylidine)acetonitrile (5b) as a white solid (50 g, 86.0%.). ¹H NMR results are shown in Table 3.

### 2-(1,1-dioxidotetra hydro-4H-thiopyran-4-ylidine) acetonitrile (5c)

To an ice cold stirred solution of 2-(tetrahydro-4H-thiopyran-4-ylidine)acetonitrile (50.0 g, 0.3591 mol, 1.0 eq.) in methanol (1500 mL) Oxone (233 g, 1.793 mol, 5.0 eq) in deionized water (1500 mL) was slowly added. A white precipitate formed during the addition. The reaction mixture was stirred at room temperature for 16h. After confirming the completion of reaction by TLC, the reaction mixture was diluted with water until solids were dissolved. The reaction mixture was then extracted with ethyl acetate (1500 mL×4). Combined organic layers were dried over sodium sulfate and evaporated under reduced pressure to afford 2-(1,1-dioxidotetrahydro-4H-thiopyran-4-ylidine)acetonitrile (5c) as a white solid (62 g, 97.7%). ¹H NMR results are shown in Table 3.

### 4-(2-aminoethyl)tetrahydro-2H-thiopyran 1,1-dioxide (4b)

To a stirred solution of 2-(1,1-dioxidotetrahydro-4H-thiopyran-4-ylidine)acetonitrile (10.0 g, 0.0584 mol, 1.0 eq) in ethanol (300 mL), aqueous ammonia (10 mL) was added at room temperature. Raney Nickel in water (15.0 g, 1.5 eq.) was added into the reaction mixture under nitrogen atmosphere. The resulting mixture was stirred under hydrogen bladder pressure for 24h at room temperature. After confirming the completion of reaction by TLC, the reaction mixture was filtered through a celite bed and the celite bed was washed with methanol (100 mL). The combined filtrate was evaporated under reduced pressure to afford 4-(2-aminoethyl)tetrahydro-2H-thiopyran-1,1-dioxide (5d) as an off white gummy solid (9.22 g, 88.8%). LCMS (ELSD) and ¹H NMR results are shown in Table 3.

### 4-(2-((2-bromo-6-Fluorobenzyl)amino)ethyl)tetrahydro-2H-thiopyran-1,1-dioxide (5f)

Compound 5f was synthesized according to a method analogous to the method described above in Example 2 for compound 4c to afford 4-(2-((2-bromo-6-fluorobenzyl)amino)ethyl) tetrahydro-2H-thiopyran-1,1-dioxide (5f) as a light brown solid (8.0 g, 63.7%.). LCMS (ELSD) and ¹H NMR results are shown in Table 3.

### N-(2-bromo-6-fluorobenzyl)-N-(2-(1,1-dioxidotetrahydro-2H-thiopyran-4-yl)-3-(trifluoromethyl)-1H-pyazole-5-carboxamide (Compound Ib)

Compound Ib was synthesized according to a method analogous to the method described above in Example 2 for compound 4d afford 4-(2-((2-bromo-6-fluorobenzyl) amino)ethyl)tetrahydro-2H-thiopyran-1,1-dioxide (Compound Ib) as a white solid (5.1 g, 44.2%.). LCMS, HPLC, and ¹H NMR results are shown in Table 4.

### Example 4

### Synthesis of Compound Ic

### Synthesis of 4-(2-((2-fluoro-5-methylbenzyl)amino)ethyl)tetrahydro-2H-thiopyran 1,1-dioxide(6c)

Compound 6c was synthesized according to a method analogous to the method described above in Example 2 for compound 4c to afford 4-(2-((2-fluoro-5-methylbenzyl) amino) ethyl) tetrahydro-2H-thiopyran-1,1-dioxide (6c) as a light brown solid (17 g, 46.01%). LCMS results are shown in Table 3.

### Synthesis of N-(2-(1,1-dioxidotetrahydro-2H-thiopyran-4-yl)ethyl)-N-(2-fluoro-5-methylbenzyl)-3-(trifluoromethyl)-1H-pyrazole-5-carboxamide (Compound Ic)

Compound Ic was synthesized according to a method analogous to the method described above in Example 2 for compound 4d to afford N-(2-(1,1-dioxidotetrahydro-2H-thiopyran-4-yl)ethyl-N-(2-fluoro-5-methyl-3-(trifluoromethyl)-1H-pyrazole-5-carboxamide (Compound Ic) as a white solid (10.3 g, 39.31%). LCMS, HPLC, and ¹H NMR results are shown in Table 4.

### Example 5

### Synthesis of Compound ID

### Synthesis of 4-(2-(((6-fluoro-1H-indol-7-yl)methyl)amino)ethyl)tetrahydro-2H-thiopyran 1,1-dioxide (7c)

Compound 7c was synthesized according to a method analogous to the method described above in Example 2 for compound 4c to afford 4-(2-((6-fluoro-1H-indole-7-yl)methyl) amino) ethyl) tetrahydro-2H-thiopyran-1,1-dioxide (7c) as a light brown solid (3.5 g, 39.14%). LCMS results are shown in Table 3.

### Synthesis of N-(2-(1,1-dioxidotetrahydro-2H-thiopyran-4-yl) ethyl)-N-((6-fluoro-1H-indol-7yl)-3-(trifluoromethyl)-1H-pyrazole-5-carboxamide (Compound Id)

Compound Id was synthesized according to a method analogous to the method described above in Example 2 for compound 4d afford N-(2-(1,1-dioxidotetrahydro-2H-thiopyran-4-yl)ethyl)-N-((6-fluoro-1H-indol-7yl)-3-(trifluoromethyl)-1H-pyrazole-5-carboxamide (Compound Id) as an off-white solid (4.2 g, 76.33%). LCMS, HPLC, and ¹H NMR results are shown in Table 4.

### Example 6

### Synthesis of Compound IE

### Synthesis of 4-(2-(((3-chloro-6-fluoro-1H-indol-7-yl) methyl) amino) ethyl) tetrahydro-2H-thiopyran-1,1-dioxide (8d)

To a stirred solution of 4-(2-(((6-fluoro-1H-indol-7-yl) methyl) amino) ethyl) tetrahydro-2H-thiopyran-1,1-dioxide (7c), (0.1 g, 0.0003 mol, 1.0 eq) in dry DCM (2.0 mL) at 10° C.-15° C., was added N-chlorosuccinimide (0.045 g, 0.00033 mol, 1.1 eq). The reaction mixture was allowed to reach room temperature and then stirred for 16 hours. After confirming the completion of reaction by TLC, the reaction mixture was diluted with DCM (10 mL). The organic layer was washed with water (10 mL×3), dried over sodium sulfate and the solvent was evaporated under reduced pressure. The crude compound obtained was purified by flash column chromatography to afford 4-(2-(((3-chloro-6-fluoro-1H-indol-7-yl) methyl) amino) ethyl) tetrahydro-2H-thiopyran-1,1-dioxide (8d) as an off-white solid (0.1 g, 90.90%). LCMS results are shown in Table 3.

### Synthesis of N-((3-chloro-6-fluoro-1H-indol-7yl)-methyl)-N-(2-(1,1-dioxidotetrahydro-2H-thiopyran-4-yl) ethyl-3-(trifluoromethyl)-1H-pyrazole-5-carboxamide (Compound Ie)

Compound Ie was synthesized according to a method analogous to the method described above in Example 2 for compound 4d to afford N-((3-chloro-6-fluoro-1H-indol-7yl)-methyl)-N-(2-(1,1-dioxidotetrahydro-2H-thiopyran-4-yl)ethyl-3-(trifluoromethyl)-1H-pyrazole-5-carboxamide (Compound Ie) as an off-white solid (0.015 g, 34.88%). LCMS, HPLC, and H NMR results are shown in Table 4.

### Example 7

### Synthesis of Compound IF

### Synthesis of 3-(((2-(1,1-dioxidotetrahydro-2H-thiopyran-4-yl) ethyl) amino)methyl)-4-fluorobenzonitrile (9c)

Compound 9c was synthesized according to a method analogous to the method described above in Example 2 for compound 4c to afford 3-(((2-(1,1-dioxidotetrahydro-2H-thiopyran-4-yl) ethyl) amino) methyl)-4-fluorobenzonitrile (9c) as a light brown solid (310 mg, 49.67%). LCMS results are shown in Table 3.

### Synthesis of N-(5-cyano-2-fluorobenzyl)-N-(2-(1,1-dioxidotetrahydro-2H-thiopyran-4-yl)ethyl)-3-(trifluoromethyl)-1H-pyrazole-5-carboxamide (9d)

Compound 9d was synthesized according to a method analogous to the method described above in Example 2 for compound 4d to afford N-(5-cyano-2-fluorobenzyl)-N-(2-(1,1-dioxidotetrahydro-2H-thiopyran-4-yl)ethyl)-3-(trifluoromethyl)-1H-pyrazole-5-carboxamide (9d) as a white solid (120 mg, 26.31%). LCMS results are shown in Table 3.

### Synthesis of N-(5-(aminomethyl)-2-fluorobenzyl)-N-(2-(1,1-dioxidotetrahydro-2H-thiopyran-4-yl)ethyl)-3-(trifluoromethyl)-1H-pyrazole-5-carboxamide (Compound If)

To a stirred solution of N-(5-cyano-2-fluorobenzyl)-N-(2-(1,1-dioxidotetrahydro-2H-thiopyran-4-yl)ethyl)-3-(trifluoromethyl)-1H-pyrazole-5-carboxamide (80 mg, 0.169 mmol, 1.0 eq) in methanol (20 mL) at room temperature, was added an aqueous solution of ammonia (0.5 mL) and 10% Pd—C(35 mg). Then the reaction mixture was stirred for 3 hours under H₂ atmosphere (balloon). After confirming the completion of reaction by TLC, the reaction mixture was cooled to room temperature and filtered through a celite bed. The celite bed was thoroughly washed with methanol (2×30 mL). The filtrate was washed with water, the organic layer was dried over sodium sulfate and solvent was evaporated under vacuum. The crude compound was purified by flash column chromatography to afford N-(5-(aminomethyl)-2-fluorobenzyl)-N-(2-(1,1-dioxidotetrahydro-2H-thiopyran-4-yl)ethyl)-3-(trifluoromethyl)-1H-pyrazole-5-carboxamide as off-white solid (15 mg, 18.75%). LCMS, HPLC, and H NMR results are shown in Table 4.

### Example 8

### Synthesis of Compound IG

### Synthesis of N-(2-(1,1-dioxidotetrahydro-2H-thiopyran-4-yl) ethyl)-N-((6-fluoro-1H-indazol-7yl)-3-(trifluoromethyl)-1H-pyrazole-5-carboxamide (Compound Ig)

Compound Ig was synthesized according to a method analogous to the method described above in Example 2 for compound 4d to afford N-(2-(1,1-dioxidotetrahydro-2H-thiopyran-4-yl) ethyl)-N-((6-fluoro-1H-indazol-7yl)-3-(trifluoromethyl)-1H-pyrazole-5-carboxamide (Compound Ig) as a white solid (0.24 g, 64.17%). LCMS, HPLC, and H NMR results are shown in Table 4.

### Example 9

### Synthesis of Compound Ih

### Synthesis of 4-(2-((2-Bromo-6-nitrobenzyl) amino) ethyl) tetrahydro-2H-thiopyran-1,1-dioxide (tic)

Compound 11c was synthesized according to a method analogous to the method described above in Example 2 for compound 4c to afford 4-(2-((2-bromo-6-nitrobenzyl)amino)ethyl)tetrahydro-2H-thiopyran-1,1-dioxide (11c) as a light brown solid (0.45 g, 52.94%). LCMS results are shown in Table 3.

### Synthesis of N-(2-bromo-6-nitrobenzyl)-N-(2-(1,1-dioxidotetrahydro-2H-thiopyran-4-yl) ethyl-3-(trifluoromethyl)-1H-pyrazole-5-carboxamide (11d)

Compound 11d was synthesized according to a method analogous to the method described above in Example 2 for compound 4d to afford N-(2-bromo-6-nitrobenzyl)-N-(2-(1,1-dioxidotetrahydro-2H-thiopyran-4-yl)ethyl-3-(trifluoromethyl)-1H-pyrazole-5-carboxamide (11d) as off-white solid (0.35 g, 55.03%). LCMS results are shown in Table 3.

### Synthesis of N-(2-amino-6-bromobenzyl)-N-(2-(1,1-dioxidotetrahydro-2H-thiopyran-4-yl) ethyl-3-(trifluoromethyl)-1H-pyrazole-5-carboxamide (Compound Ih)

Compound Ih was synthesized according to a method analogous to the method described above in Example 2 for compound Ia to afford N-(2-amino-6-bromobenzyl)-N-(2-(1,1-dioxidotetrahydro-2H-thiopyran-4-yl)-ethyl-3-(trifluoromethyl)-1H-pyrazole-5-carboxamide (Compound Ih) as a white solid (0.07 g, 49.64%). LCMS, HPLC, and H NMR results are shown in Table 4.

### Example 10

### Synthesis of Compound Ii

### Synthesis of 4-(2-((2-nitrobenzyl) amino)ethyl)tetrahydro-2H-thiopyran 1,1-dioxide(12c)

Compound 12c was synthesized according to a method analogous to the method described above in Example 2 for compound 4c to afford 4-(2-((2-nitrobenzyl)amino)ethyl)tetrahydro-2H-thiopyran 1,1-dioxide (12c) as a light brown solid (530 mg, 51.30%). LCMS results are shown in Table 3.

### Synthesis of N-(2-(1,1-dioxidotetrahydro-2H-thiopyran-4-yl)ethyl)-N-(2-nitrobenzyl)-3-(trifluoromethyl)-1H-pyrazole-5-carboxamide (12d)

Compound 12d was synthesized according to a method analogous to the method described above in Example 2 for compound 4 to afford N-(2-(1,1-dioxidotetrahydro-2H-thiopyran-4-yl)ethyl)-N-(2-nitrobenzyl)-3-(trifluoromethyl)-1H-pyrazole-5-carboxamide (12d) as a white solid (630 mg, 83%). LCMS results are shown in Table 3.

### Synthesis of N-(2-aminobenzyl)-N-(2-(1,1-dioxidotetrahydro-2H-thiopyran-4-yl)ethyl)-3-(trifluoromethyl)-1H-pyrazole-5-carboxamide (Compound Ii)

To a stirred solution of N-(2-(1,1-dioxidotetrahydro-2H-thiopyran-4-yl)ethyl)-N-(2-nitrobenzyl)-3-(trifluoromethyl)-1H-pyrazole-5-carboxamide (12d; 400 mg, 0.843 mmol, 1.0 eq) in ethyl acetate (10 mL) at room temperature, was added 10% Pd/C (200 mg). Then the reaction mixture was stirred for 3 hours under H2 atmosphere (balloon). After confirming the completion of reaction by TLC, the reaction mixture was cooled to room temperature and filtered through a celite bed. The celite bed was thoroughly washed with methanol (2×30 mL). The filtrate was washed with water, the organic layer was dried over sodium sulfate, and the solvent was evaporated under vacuum. The crude compound was purified by flash column chromatography to afford N-(2-aminobenzyl)-N-(2-(1,1-dioxidotetrahydro-2H-thiopyran-4-yl)ethyl)-3-(trifluoromethyl)-1H-pyrazole-5 carboxamide) (Compound Ii) as an off-white solid (180 mg, 48.12%). LCMS, HPLC, and ¹H NMR results are shown in Table 4.

### Example 11

### Synthesis of Compound Ij

### Synthesis of 4-(2-((2-chloro-6-fluoro benzyl) amino) ethyl) tetrahydro-2H-thiopyran-1,1-dioxide (13c)

Compound 13c was synthesized according to a method analogous to the method described above in Example 2 for compound 4c to afford 4-(2-((2-chloro-6-fluoro benzyl) amino) ethyl) tetrahydro-2H-thiopyran-1,1-dioxide (13c) as a light brown gummy solid (0.11 g, 27.90%). LCMS results are shown in Table 3.

### N-(2-chloro-6-fluoro benzyl)-N-(2-(1,1-dioxidotetrahydro-2H-thiopyran-4-yl) ethyl-3-(trifluoromethyl)-1H-pyrazole-5-carboxamide (Ij)

Compound Ij was synthesized according to a method analogous to the method described above in Example 2 for compound 4d to afford N-(2-chloro-6-fluorobenzyl)-N-(2-(1,1-dioxidotetrahydro-2H-thiopyran-4-yl)ethyl-3-(trifluoromethyl)-1H-pyrazole-5-carboxamide (Compound Ij) as an off-white solid (0.035 g, 23.48%). LCMS, HPLC, and ¹H NMR results are shown in Table 4.

U.S. Provisional Patent Application No. 62/822,700, filed Mar. 22, 2019, to which the present application claims priority, is hereby incorporated herein by reference in its entirety. From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

TABLE 3

| | | LCMS |
|---|---|---|
| | | Column |
| Cmpd | ¹H NMR | Retention time (min) |
| 4c | | Atlantis C₁₈ (50 × 4.6 mm) 5 |
| | | μm 1.038 min. [M + H]⁺: |
| | | 347.0 |
| 4d | | Atlantis C₁₈ (50 × 4.6 mm) 5 |
| | | μm 2.712 min.; [M + H]⁺: |
| | | 509.0 |
| 5b | (400 MHz, CDCl₃): δ 5.17 (s, 1H), | |
| | 2.85-2.88 (m, 2H), 2.74-2.80 (m, | |
| | 4H), and 2.60-2.62 (m, 2H). | |
| 5c | (400 MHz, CDCl₃): δ 5.40 (s, 1H), | |
| | 3.12-3.19 (m, 6H), and 2.90-2.93 | |
| | (m, 2H). | |
| 4b | (400 MHz, DMSO-d₆): δ 3.25-3.34 | Atlantis C₁₈ (50 × 4.6 mm) |
| | (bs, 2H), 2.97-3.17 (m, 4H), 2.47- | 5 μm 0.675 min; [M + H]⁺: |
| | 2.64 (m, 2H), 1.96-1.99 (m, 2H), | 177.8 |
| | 1.45-1.72 (m, 3H), and 1.28-1.39 | |
| | (m, 2H) | |
| 5f | (400 MHz, DMSO-d₆): δ 7.48 (dd, | Atlantis C₁₈ (50 × 4.6 mm) 5 |
| | J = 6.72, 1.2 Hz, 1H), 7.22-7.31 | μm 1.076 min; [M + H]⁺: |
| | (m, 2H), 3.81 (d, J = 1.84 Hz, | 363.8 |
| | 2H), 2.95-3.18 (m, 5H), 2.49-2.51 | |
| | (m, 1H), 1.82-1.99 (d, 2H), 1.82 | |
| | (s, 1H), 1.67-1.51 (m, 3H), and | |
| | 1.41-1.18 (m, 2H). | |
| 6C | | Atlantis C18 (50 × 4.6 mm) 5 |
| | | μm 1.106 min. [M + H]⁺: |
| | | 300.0 |
| 7C | | Atlantis C₁₈ (50 × 4.6 mm) 5 |
| [Ex. 5] | | μm 1.869 min. [M + H]⁺: |
| | | 324.8 |
| 7c | | Atlantis C18 (50 × 4.6 mm) 5 |
| [Ex. 6] | | μm 1.771 min. [M − H]+: |
| | | 322.5 |
| 8d | | Atlantis C₁₈ (50 × 4.6 mm) 5 |
| | | μm 1.41 min.; [M + H]⁺: |
| | | 359.1 |
| 9c | | Atlantis C₁₈ (50 × 4.6 mm) 5 |
| | | μm 0.952; (M + H)⁺: 311.1. |
| 9d | | ZORBAX XDB C₁₈ (50 × |
| [Ex. 7] | | 4.6 mm) 3.5 μm |
| | | 2.503; (M + H)⁺: 471.1 |
| 9d | | Atlantis C₁₈ (50 × 4.6 mm) 5 |
| [Ex. 8] | | μm 1.505 min. [M + H]⁺: |
| | | 326.1 |
| 11c | | Atlantis C₁₈ (50 × 4.6 mm) 5 |
| | | μm 1.558 min. [M + H]⁺: |
| | | 393.0 |
| 11d | | Atlantis C₁₈ (50 × 4.6 mm) 5 |
| | | μm 2.761 min.; [M − H]⁺: |
| | | 550.7 |
| 12c | | Atlantis C₁₈ (50 × 4.6) 5 μm |
| | | 1.435; (M + H)⁺: 313.2. |
| 12d | | Atlantis C₁₈ (50 × 4.6) 5 μm |
| | | 2.546; (M − H)⁺: 473.1 |
| 13c | | Atlantis C₁₈ (50 × 4.6 mm) 5 |
| | | μm 1.868 min. [M + H]⁺: |
| | | 321.1 |

Note:
For all of the above LCMS results, the following conditions apply:
Mobile phase A: 0.1% formic acid in H20: acetonitrile (95:5)
Mobile phase B: acetonitrile
Flow: 1.5 mL/min

TABLE 4

| | HPLC | | |
|---|---|---|---|
| | Column | | |
| | Mobile phase B | | LCMS |
| | Flow | | Column |
| | Retention time (min) | | Mobile phase A |
| Cmpd | Purity (max) | ¹H NMR | Retention time (min) |
| Ia | XBridge C₈ (50 × | (400 MHz, CD₃OD): δ 7.08-7.04 (m, 2H), | Atlantis C₁₈ (50 × 4.6 mm) 5 μm |
| | 4.6 mm, 3.5 μm) | 6.74-6.67 (m, 2H), 5.20 (s, 2H), 3.65-3.5 (m, | 0.1% formic acid in |
| | 0.1% | 2H), 3.06-2.95 (m, 4H), 2.03-1.67 (m, 2H), | H₂O: acetonitrile (95:5) |
| | TFA in acetonitrile | 1.64-1.53 (m, 5H) | 2.715 min; [M + H]⁺: 479.0 |
| | 2.0 mL/min | | |
| | 4.145 min | | |
| | 95.13% | | |
| Ib | XBridge C₈ (50 × | (400 MHz, CD₃OD): δ 7.51 (d = 7.6 Hz, | ZORBAX XDB C₁₈ (50 × |
| | 4.6 mm, 3.41 m) | 1H), 7.37-7.31 (m, 1H), 7.23-7.19 (m, 1H), | 4.6 mm) 3.5 μm |
| | 0.1% | 7.10-6.99 (m, 1H), 5.10 (s, 2H), 3.49-3.40 | 0.1% formic acid in |
| | TFA in acetonitrile | 2H), 3.06-2.97 (m, 5H), 2.05-1.87 (m, 2H), | H₂O: acetonitrile (95:5) |
| | 2.0 mL/min | and 1.67-1.48 (m, 5H) | 2.672 min; [M + H]⁺: 526.0 |
| | 4.428 min | | |
| | 99.44% | | |
| Ic | XBridge C₈ (50 × | (400 MHz, CD₃OD):, δ 7.18 (t, J = 22.00 Hz, | ZORBAX XDB C₁₈ (50 × |
| | 4.6 mm, 3.5 μm) | 2H), 7.03 (t, J = 9.20 Hz, 2H), 6.77 (s, 1H), | 4.6 mm) 3.5 μm |
| | acetonitrile | 4.83 (s, 2H), 3.33 (t, J = 1.60 Hz, 2H), 3.06- | 0.1% formic acid in |
| | 2.0 mL/min | 2.95 (m, 5H), 2.38-2.32 (m, 4H), 2.03-1.96 | H₂O: acetonitrile (95:5) |
| | 4.544 min | (s, 3H), 1.74-1.62 (m, 3H). | 2.602 min; [M + H]⁺: 462.1 |
| | 98.18% | | |
| Id | XBridge C₈ (50 × | 400 MHz, CD₃OD): δ 7.54-7.57 (m, 1H), | Atlantis C₁₈ (50 × 4.6 mm) 5 μm |
| | 4.6 mm, 3.5 μm) | 7.32 (s, 1H), 7.11 (s, 1H), 6.90 (t, J = 8.80 Hz, | 0.1% formic acid in |
| | 0.1% | 1H), 6.51 (s, 1H), 5.12 (s, 2H), 3.48-3.53 (m, | H₂O: acetonitrile (95:5) |
| | TFA in acetonitrile | 1H), 2.97 (d, J = 12.40 Hz, 4H), 2.03 (s, 2H), | 2.721 min.; [M − H]⁺: 485.1 |
| | 2.0 mL/min | 1.88 (s, 3H), 1.50 (s, 2H). | |
| | 4.661 min | | |
| | 97.23% | | |
| Ie | XBridge C₈ | (400 MHz, CD₃OD): δ 7.54 (t, J = 4.88 Hz, | Atlantis C₁₈ (50 × 4.6 mm) 5 μm. |
| | (50 × 4.6 mm, 3.5 μm) | 1H), 7.36 (s, 1H), 7.02 (t, J = 11.44 Hz, 2H), | 0.1% formic acid in |
| | acetonitrile | 5.10 (s, 2H), 3.58 (s, 2H), 2.99-3.02 (m, 5H) | H₂O acetonitrile (95:5) |
| | 2.0 mL/min | 1.91 (s, 3H), 1.50-1.57 (m, 7H), 1.31-1.38 (m | 2.91 min.; [M − H]⁺: 519.0 |
| | 5.09 min | 2H) | |
| | 98.12% | | |
| If | XBridge C₈ (50 × | (400 MHz, DMSO-d₆): δ 7.32-7.59 (m, 3H), | ZORBAX XDB C₁₈ (50 × |
| | 4.6 mm, 3.5 μm) | 6.69-6.88 (m, 1H), 4.88-5.04 (m, 2H), 3.93 (s, | 4.6 mm) 3.5 μm |
| | acetonitrile | 2H), 3.40-3.64 (m, 2H), 3.02 (t, J = 12.00 Hz, | 0.1% formic acid in |
| | 2.0 mL/min | 4H), 1.91-2.09 (m, 3H), 1.30-1.57 (m, 4H). | H₂O: acetonitrile (95:5) |
| | 2.971 min | | 1.847; (M + H)⁺: 477.2 |
| | 94.44% | | |
| Ig | XBridge C₈ (50 × | (400 MHz, CD₃OD): δ 8.13 (d, J = 12.28 Hz, | Atlantis C₁₈ (50 × 4.6 mm) 5 μm |
| | 4.6 mm, 3.5 μm) | 1H), 7.83 (s, 1H), 7.00-7.09 (m, 2H), 5.14 (s, | 0.1% formic acid in |
| | acetonitrile | 2H), 3.60 (s, 1H), 2.83-3.02 (m, 4H), 1.73- | H₂O acetonitrile (95:5) |
| | 2.0 mL/min | 2.04 (m, 2H), 1.67 (d, J = 25.44 Hz, 3H), 1.55 | 2.598 min.; [M + H]⁺: 488.1 |
| | 3.993 min | (t, J = 10.56 Hz, 2H) | |
| | 94.08% | | |
| Ih | XBridge C₈ (50 × | (400 MHz, CD₃OD): δ 6.99 (t, J = 7.80 Hz, | Atlantis C₁₈ (50 × 4.6 mm) 5 μm |
| | 4.6 mm, 3.5 μm) | 2H), 6.91 (d, J = 7.68 Hz, 1H), 6.71 (d, J = | 0.1% formic acid in H₂O: |
| | acetonitrile | 7.16 Hz, 1H), 5.06 (s, 2H), 3.55 (s, 2H), 2.92- | acetonitrile (95:5) |
| | 2.0 mL/min | 3.06 (m, 4H), 1.92 (s, 2H), 1.45-1.57 (m, 2H) | 2.681 min; [M + H]⁺: 523.0 |
| | 4.241 min | | |
| | 95.77% | | |
| Ii | XBridge C₈ (50 × | (400 MHz, DMSO-d₆): δ 6.69-7.11 (m, 4H), | Atlantis C₁₈ (50 × 4.6 mm) 5 μm |
| | 4.6 mm, 3.5 μm) | 4.75 (s, 2H), 3.52 (t, J = 6.80 Hz, 2H), 3.01 (s, | 0.1% formic acid in H₂O |
| | acetonitrile | 4H), 1.87-2.14 (m, 2H), 1.20-1.67 (m, 6H) | 2.392; (M + H)⁺: 445.1 |
| | 2.0 mL/min | | |
| | 3.125 min | | |
| | Purity (max): | | |
| | 97.49% | | |
| Ij | Atlantis C₁₈ (250 × | (400 MHz, CD₃OD): δ 7.39-7.44 (m, 1H), | Atlantis C₁₈ (50 × 4.6) 5 μm |
| | 4.6 mm) 5 μm | 7.33 (d, J = 8.00 Hz, 1H), 7.18 (t, J = 8.80 Hz, | 0.1% formic acid in |
| | acetonitrile | 1H), 7.05 (s, 1H), 5.10 (d, J = 40.40 Hz, 2H), | H₂O acetonitrile (95:5) |
| | 1.0 mL/min | 3.62 (s, 2H), 2.88-3.08 (m, 4H), 1.84-2.07 (m, | 2.596 min.; [M + H]⁺: 482.1 |
| | 12.576 min | 2H), 1.52-1.67 (m, 5H) | |
| | 97.94% | | |

Note:
For all of the above LCMS results, the following conditions apply:
Mobile phase A: 0.1% TFA in H2O
For all of the above LCMS results, the following conditions apply:
Mobile phase B: acetonitrile
Flow: 1.5 mL/min

1. A compound having the following structure (I): 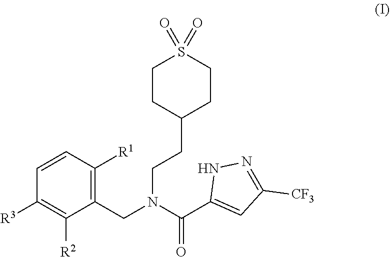
or a pharmaceutically acceptable salt, isotopic form, tautomer or prodrug thereof,
wherein:
R¹ is F or NH₂,
R² is H, Cl or Br; or R² joins with R³ to form an optionally substituted heteroaryl; and
R³ is H or optionally substituted C1-C6 alkyl; or R³ joins with R² to form an optionally substituted heteroaryl.

2. The compound of claim 1, wherein:
R¹ is F or NH₂,
R² is H, Cl or Br; and
R³ is H or optionally substituted C1-C6 alkyl.

3. The compound of claim 1, wherein R³ is H.

4. The compound of claim 1, wherein R³ is methyl or aminomethyl.

5. The compound of claim 1, wherein R² is H.

6. The compound of claim 1, wherein:
R¹ is F or NH₂,
R² is Cl or Br; and
R³ is H.

7. The compound of claim 6, wherein R¹ is F.

8. The compound of claim 6, wherein R¹ is NH₂.

9. The compound of claim 6, wherein R² is Cl.

10. The compound of claim 6, wherein R² is Br.

11. The compound of claim 1, wherein R² and R³ join to form a 5-membered optionally substituted heteroaryl.

12. The compound of claim 11, wherein R¹ is F.

13. The compound of claim 11, wherein R¹ is NH₂.

14. A compound having one of the following structures (Ia), (Ib), (Ic), (Id), (Ie), (If), (Ig), (Ih), (Ii), or (Ij): 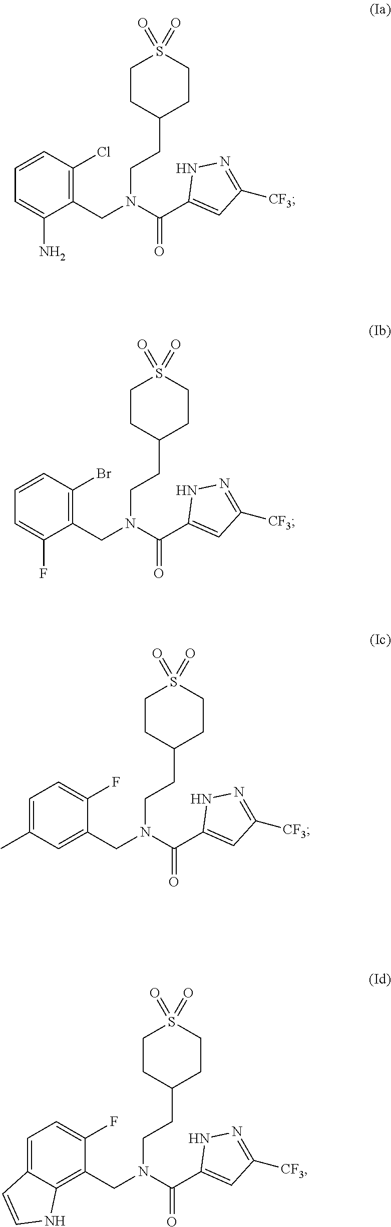 [SMILES: Nc1cccc(Cl)c1CN(CCC1CCS(=O)(=O)CC1)C(=O)c1cc(C(F)(F)F)n[nH]1 ; O=C(c1cc(C(F)(F)F)n[nH]1)N(CCC1CCS(=O)(=O)CC1)Cc1c(F)cccc1Br ; Cc1ccc(F)c(CN(CCC2CCS(=O)(=O)CC2)C(=O)c2cc(C(F)(F)F)n[nH]2)c1 ; O=C(c1cc(C(F)(F)F)n[nH]1)N(CCC1CCS(=O)(=O)CC1)Cc1c(F)ccc2cc[nH]c12] 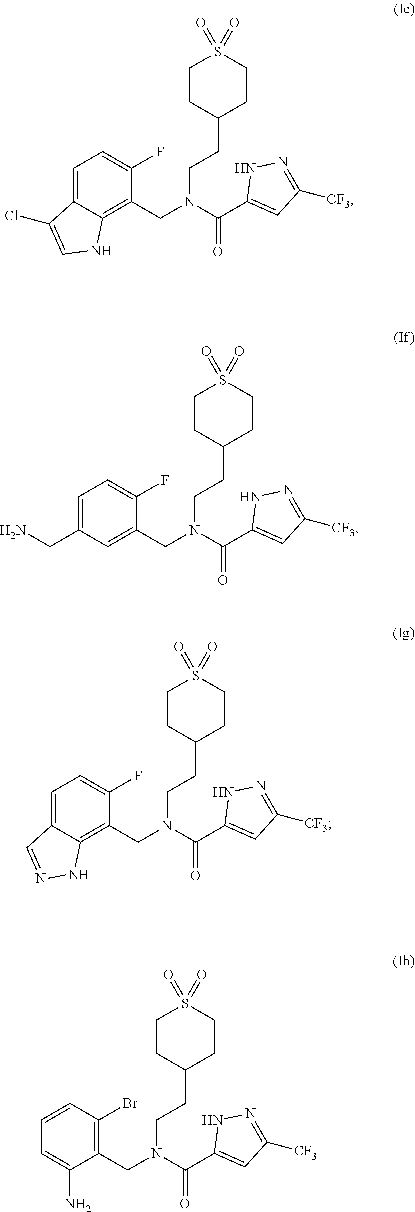 [SMILES: O=C(c1cc(C(F)(F)F)n[nH]1)N(CCC1CCS(=O)(=O)CC1)Cc1c(F)ccc2c(Cl)c[nH]c12 ; NCc1ccc(F)c(CN(CCC2CCS(=O)(=O)CC2)C(=O)c2cc(C(F)(F)F)n[nH]2)c1 ; O=C(c1cc(C(F)(F)F)n[nH]1)N(CCC1CCS(=O)(=O)CC1)Cc1c(F)ccc2cn[nH]c12 ; Nc1cccc(Br)c1CN(CCC1CCS(=O)(=O)CC1)C(=O)c1cc(C(F)(F)F)n[nH]1] 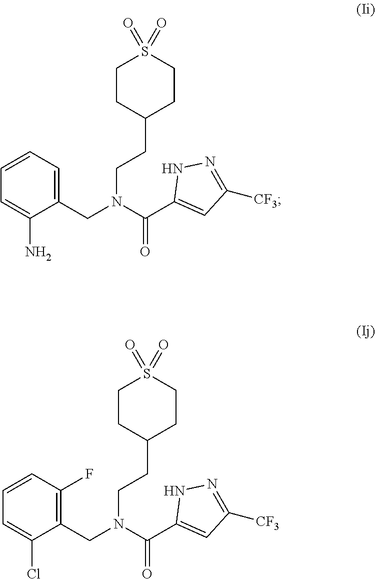 [SMILES: Nc1ccccc1CN(CCC1CCS(=O)(=O)CC1)C(=O)c1cc(C(F)(F)F)n[nH]1 ; O=C(c1cc(C(F)(F)F)n[nH]1)N(CCC1CCS(=O)(=O)CC1)Cc1c(F)cccc1Cl]
or a pharmaceutically acceptable salt, isotopic form, tautomer or prodrug thereof.

15. A pharmaceutical composition comprising the compound of claim 1, or a pharmaceutically acceptable salt, isotopic form, tautomer or prodrug thereof, and a pharmaceutically acceptable carrier or excipient.

16. A method for modulating pyruvate kinase muscle isozyme M2 (PKM2) activity in a subject in need thereof, the method comprising administering a therapeutically effective amount of the composition of claim 15 to the subject.

17. A method for treating cancer in a subject in need thereof, the method comprising administering a therapeutically effective amount of the composition of claim 15 to the subject.

18. The method of claim 17, wherein the cancer is an advanced solid tumor resistant to treatment with an immuno-oncology (IO) agent and the cancer is NPM-ALK anaplastic large cell lymphoma.

19. The method of claim 17, wherein the cancer is an EGFR-mutant non-small cell lung cancer resistant to treatment with a tyrosine kinase inhibitor.

20. The method of claim 17, further comprising administering, or instructing the administration of, a diet low in serine and/or glycine to the subject.
